(19) United States
(12) Patent Application Publication
(10) Pub. No.: US 2023/0165873 A1
(43) Pub. Date: Jun. 1, 2023

(54) Methods of Use for Single Molecule Compounds Providing Multi-Target Inhibition to Treat Covid 19

(71) Applicant: SignalRx Pharmaceuticals, Inc., Cumming, GA (US)

(72) Inventors: Donald L. Durden, Charlotte, NC (US); Guillermo A. Morales, Oro Valley, AZ (US); Joseph R. Garlich, Cape Coral, FL (US)

(73) Assignee: SignalRx Pharmaceuticals, Inc., Cumming, GA (US)

(21) Appl. No.: 17/922,045

(22) PCT Filed: Apr. 28, 2021
(86) PCT No.: PCT/US2021/029550

§ 371 Date: Oct. 28, 2022

Related U.S. Application Data
(60) Provisional application No. 63/017,191, filed on Apr. 29, 2020.

(51) Int. Cl. A61K 31/5377; A61K 45/06; A61P 31/14; A61K 31/496; A61K 31/519; A61K 31/538

(52) U.S. Cl. CPC A61K 31/5377; A61K 31/496; A61K 31/519; A61K 31/538; A61K 45/06; A61P 31/14

(57) ABSTRACT

The invention relates to compounds useful for inhibiting at least one member of the BET family and at least one kinase such as but not limited to mTOR, and to methods of treating diseases including COVID-19 by administration of a compound(s) of Formulas I-V or pharmaceutically acceptable salts thereof as defined herein.

### FIELD OF THE INVENTION

The present invention relates to thienopyranone and furanopyranone compounds and methods of treating diseases in mammals including humans by administering a compound(s) of the invention. In one aspect of the invention, one or more compounds are administered to provide therapeutic benefit for a patient suffering from a coronavirus infection.

### BACKGROUND

Coronaviruses are a large family of viruses that usually cause mild to moderate upper-repiratory tract illnesses such as the common cold. However, three new coronaviruses have emerged from animal populations during the past two decades to cause serious and widespread illness and death. There are hundreds of known coronaviruses most of which are confined to animals such as pigs, camels, bats, and cats. In some instances, however, coronaviruses can jump to humans (i.e., spillover event). Of the seven known coronaviruses that have stricken humans, four cause only mild to moderate disease. However, three of the coronaviruses can cause more serious, even fatal, disease. SARS coronavirus (SARS-CoV) emerged in November 2002 and caused sever acute respiratory syndrome (SARS). That virus disappeared by 2004. Middle East respiratory syndrome (MERS) is caused by the MERS coronavirus (MERS-CoV). MERS was first identified in September 2012 after emerging from a camel reservoir. The third novel coronavirus to emerge in this century is called SARS-CoV-2. It causes coronavirus disease 2019 (i.e., COVID-19) which first emerged in China in December 2019, and was declared a global pandemic by the World Health Organization on Mar. 11, 2020.

There is great interest in finding molecules that will prevent or treat COVID-19 infections. A database of around 50,000 molecules with antiviral activity has been compiled (https://cen.acs.org/acs-news/publishing/CAS-curates-dataset-antiviral-compound/98/web/2020/04). A more focused approach to finding potential molecular targets has been described by Gordon et al. [David E. Gordon, et al., A SARS-CoV-2-Human Protein-Protein Interaction Map Reveals Drug Targets and Potential Drug-Repurposing. bioRxiv, 2020: p. 2020.03.22.002386]. A proteomic platform was used to identify the interactions of 26 of 29 COVID-19 proteins with cellular targets in human cells and determined 67 interactions as potential targets for drug development. Two of the key targets identified - BRD2 and BRD4, belong to the BET bromodomain family. Another key target identified was the mTOR protein kinase. BRD2/BRD4 proteins were noted to bind to the CoV envelope protein (E). It is suggested that BRD2/BRD4 interaction with E protein disrupts bromodomain protein interaction with the human histone code to selectively disrupt mammalian transcription in CoV infected human cells to promote the intracellular survival of the virus. mTOR is bound to the CoV nucleoprotein (N). These data suggest important interactions between the SARS-CoV-2 virion and elements of the host machinery that may be critical for viral pathogenesis.

PI3 kinases are a large family of lipid kinases comprising roughly 16 members divided into 3 classes based on sequence homology and the particular product formed by enzyme catalysis. The class I PI3 kinases are composed of 2 subunits: a 110 kd catalytic subunit and an 85 kd regulatory subunit. Class I PI-3 kinases are involved in important signal transduction events downstream of cytokines, integrins, growth factors and immunoreceptors, and control of this pathway may lead to important therapeutic effects. Inhibition of class I PI3 kinase induces apoptosis, blocks tumor induced angiogenesis in vivo, and increases radiosensitivity in certain tumors.

Molecular and genetic studies have demonstrated a strong correlation between the PI3 kinase pathway (also known as PI3K-AKT pathway) and a variety of diseases in humans such as inflammation, autoimmune conditions, and cancers (P. Workman et al., Nat. Biotechnol. 2006, 24, 794-796). The PI3 kinase pathway controls a number of cellular functions including cell growth, metabolism, differentiation, and apoptosis.

The PI3 kinase pathway comprises a number of enzymes including PI3 kinase, PTEN (Phosphatase and Tensin homolog deleted on chromosome 10), and AKT (a serine/threonine kinase) all of which are involved in producing and maintaining intracellular levels of second messenger molecule PtdIns(3,4,5)P3 (Phosphatidylinositol (3,4,5)-trisphosphate or PIP3). Homeostasis in the levels of this important second messenger is maintained by the interaction between PI3 kinase and PTEN. When either PI3 kinase or PTEN are mutated and/or reduced in activity PIP3 levels are perturbed which may act as a trigger in the development of diseases including but not limited to cancer.

Genetic and biochemical evidence from several animal models has established that constitutive levels of AKT in the PI3 kinase pathway can regulate TOR (mTOR in mammalian systems) through phosphorylation of the tuberous sclerosis complex (K. Inoki et al., Nat. Cell Biol. 2002, 4, 648- 657). For example, tumors with loss-of-function mutations in PTEN exhibit constitutive activation of AKT ands mTOR. Thus, mTOR as a kinase can be inhibited directly or by inhibition of the upstream pathway elements such as PI3 kinase.

In addition, a growing list of diseases have been correlated with epigenetic changes in gene expression rather than by mutations in DNA nucleotide sequence. Epigenetic effects can be controlled by three types of proteins: the writers (i.e., DNA methyltransferase which adds methyl groups to DNA), the erasers (i.e., histone deacetylase, HDAC, which removes acetyl groups from histones), and the readers (i.e., BET bromodomain proteins such as BRD2, BRD3, BRD4 and BRDT). Bromodomain proteins recruit regulatory enzymes such as “writers” and “erasers” which lead to regulation of gene expression. Inhibitors of bromodomain proteins are potentially useful in the treatment of a variety of diseases including obesity, inflammation, and cancer (A.C. Belkina et al., Nat. Rev. Cancer 2012, 12, 465-477).

Given that BET proteins BRD2 and BRD4 may be potential targets for COVID-19 drug development (See supra), there is interest in identifying BET inhibitors as potential therapeutics for COVID-19 treatment. BET inhibitors act as acetylated lysine mimetics that disrupt the binding interaction of BET proteins with acetylated lysine residues on histones (D.S. Hewings et al., J. Med. Chem. 2012, 55, 9393-9413).

COVID-19 patients are at risk for developing a number of adverse complications including a potentially fatal acute respiratory distress syndrome (ARDS) (Cell Reports Medicine 2020 Nov 17;1(8): 100145; https://doi.org/10.1016/j.xcrm.2020.100145; PMID: 33225317); cytokine storm, severe systemic capillary leak syndrome, thromboembolic events, and multi-organ dysfunction/failure. Spleen Tyrosine Kinase (SYK) is a master regulator of signal transduction pathways that are implicated in these COVID-19 associated complications, which involve hyperactivation of both innate and acquired immune systems (Blood (2020) 136 (Supplement 1): 35.; https://doi.org/10.1182/blood-2020-141045). SYK inhibition reduces the level of mucin-1 (MUC1), a molecule associated with acute lung injury (ALI) and acute respiratory distress syndrome (ARDS). Therefore, there is interest in identifying pharmaceutical agents that inhibit SYK for the prevention and/or treatment of COVID-19 and/or complications thereof.

The present invention provides compounds including Compound 0 (“Compd 0”; shown below) and analogs thereof for the prevention and/or treatment of COVID-19 infections and complications thereof including but not limited to lung pathology such as lung fibrosis or pulmonary fibrosis. Compound 0 has been demonstrated to be a potent dual inhibitor of BRD4 and PI3K with less toxicity than the combination of two single inhibitors (e.g., a PI3K inhibitor plus a separate BRD4 inhibitior) (see U.S. Pat. 8,557,807, U.S. Pat. 9,505,780, Morales et al., J. Med. Chem. 2013, and Andrews et al., PNAS Feb. 14, 2017, vol. 114, no. 7, pp E1072-E108; the entire contents of which are herein incorporated by reference).

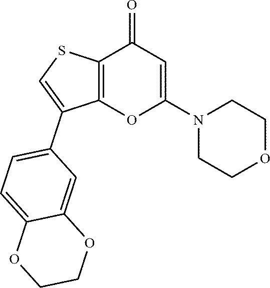 [SMILES: O=c1cc(N2CCOCC2)oc2c(-c3ccc4c(c3)OCCO4)csc12]

Off-target toxicities represent a major hurdle when administering multiple single molecule inhibitors. A more nuanced approach involves administering multi-target single molecule inhibitors which are potentially advantageous over combinations of single-target inhibitors for a number of reasons including: a) simpler straightforward clinical development, b) reduced development costs; c) lower toxicity; d) lower non-target side effects due to non-target drug interactions; e) wider therapeutic index, e) simultaneous target inhibition to provide greater efficacy (versus combinations of agents suffering from differing ADME dynamics); f) lower financial costs to patients and the healthcare system; g) increased efficacy and longer durations of response; and h) accelerated drug development.

Single-molecule, multi-target inhibition can avoid some of the problems arising from differing ADME properties associated with administering separate agents such as dose limiting toxicity resulting from additive off-target toxicities of the individual drugs. In addition, a single molecule, multi-target inhibitor could dramatically simplify taking medications and improve patient compliance. For example, a patient whose treatment includes inhibition of multiple targets would generally take separate medicines to achieve inhibition of each target, whereas a single molecule, multitarget inhibitor could achieve the same objective with just a single medication.

In some instances, COVID-19 infections result in pulmonary fibrosis which may require hospitalization and ventilation and unfortunately has a high incidence of mortality. Pulmonary fibrosis is characterized by scarring in the lungs that reduces oxygen flow. Pulmonary fibrosis resulting from COVID-19 prevents a patient from receiving enough oxygen needed for survival and/or recovery. Fibrosis is the formation of excess fibrous connective tissue in an organ or tissue in a reparative or reactive process. Several signaling pathways contribute to the development of both fibrosis and lung cancer. The phosphoinositide 3-kinase (PI3K) pathway is activated in both pulmonary cancer (Annu Rev Pathol 2009; 4: 127-150) and idiopathic pulmonary fibrosis (IPF) (Thorax 2016; 71:701-711 & Scientific Reports 2017; 7:14272). Additionally PI3K inhibition has proven effective against IPF in in vivo preclinical studies (Am J Pathol 2010;176:679-686) along with promising results of PI3K inhibition in a Phase 1 clinical trial for IPF administered orally (European Respiratory Journal 2019 (in press); see https://erj.ersjournals.com/content/early/2018/12/14/13993003.01992-2018). The bromodomain 4 (BRD4) pathway is also reported to drive IPF pathology (Am J Pathol 2013, 183:470-479) and the inhibition of BRD4 is reported to inhibit fibrosis in bleomycin-induced lung fibrosis models (Am J Pathol 2013, 183:470-479; Mol Pharmacol 2013;83:283-293 and Am J Respir Crit Care Med 2019;199:A5879). Further, BRD4 inhibition in an aging model of lung fibrosis reduced markers of fibrosis (Am J Respir Crit Care Med 2019; 199:A5879). Lastly, several studies and reviews have highlighted similarities between IPF and cancer (Thorax 2016;71:675-676). Therefore, compounds of the present invention are expected to provide therapeutic benefit against COVID-19 and complications thereof including, but not limited to, pulmonary fibrosis, IPF, and scleroderma, by inhibiting a BET protein, such as BRD4, and a kinase such as PI3K and/or mTOR.

Thus, there is a need for effective treatments to prevent and/or treat infections from Coronaviruses such as SARS coronavirus (SARS-CoV), MERS-CoV, and SARS-CoV-2 leading to the disease COVID-19, as well as complications arising therefrom including, but not limited to, acute respiratory failure, pneumonia, acute respiratory distress syndrome, acute liver injury, acute cardiac injury, acute kidney injury, septic shock, disseminated intravascular coagulation, and rhabdomylosis. Compounds of the invention provide single molecule capability of potently inhibiting at least one member of the BET family and at least one kinase including but not limited to mTOR and PI3K to inhibit multiple targets of the virus.

### SUMMARY OF THE INVENTION

The present invention relates to thienopyranone and furanopyranone compounds that are useful for inhibiting at least one member of the BET family and at least one kinase such as but not limited to mTOR for the treatment or prevention of infection by coronaviruses including but not limited to SARS-CoV-2 and the resulting disease COVID-19.

In particular, the present invention relates to thienopyranone and furanopyranone compounds, conjugates, and pharmaceutical compositions thereof, and use of the compounds as therapeutic agents in mammals. Some of the compounds disclosed in this application can be prepared by methods described in U.S. Pat. 8,557,807 and 9,505,780, WO 2018/140730, WO 2018/226739, WO 2020/023340, and in Morales et al., J. Med. Chem. 2013, the entire contents of which are herein incorporated by reference.

In one aspect, the present invention relates to methods for treating diseases in mammals including humans by administering a therapeutically effective amount of a compound of Formula I or a pharmaceutically acceptable salt thereof:

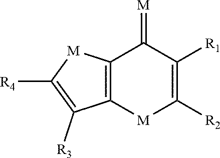

wherein M is independently oxygen (O) or sulfur (S);
R1 is selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from R1, morpholine, thiomorpholine, or piperazine;
R3 is selected from R1;
R4 is selected from R1; and
where R1-R4 may independently contain varying amounts of isotopic substitution.

These and other objects of the invention are evidenced by the summary of the invention, the description of the preferred embodiments and the claims.

### BRIEF DESCRIPTION OF THE DRAWINGS

FIG. 1 shows a graph of the effect of compound 0 in blocking COVID-19 infectivity of human Hela-ACE2 transduced cells in vitro.

FIGS. 2A-2C show lung tissue sections stained with Masson Trichrome from mice carrying the Fli1 deletion and treated with PBS (2A), Bleomycin (2B), or Bleomycin plus compound 0 (2C).

FIG. 3 shows collagen content in lung tissue from mice treated with PBS, Bleomycin, or Bleomycin plus compound 0.

FIG. 4 shows Ascroft scores in lung tissue from mice treated with PBS, Bleomycin, or Bleomycin plus inhibitor.

FIG. 5 shows fold induction of COL1A2 in cultured fibroblasts from 6 scleroderma patients treated with compound 0.

FIG. 6 shows fold induction of COL1A2 in cultured fibroblasts from 6 scleroderma patients treated with IRA-1 inhibitor NT 157.

### DETAILED DESCRIPTION

### A. Definitions

As used herein, coronaviruses are a large family of viruses that usually cause mild to moderate upper-repiratory tract illnesses such as the common cold. However, three new coronaviruses have emerged from animal populations during the past two decades to cause serious and widespread illness and death. There are hundreds of known coronaviruses most of which are confined to animals such as pigs, camels, bats, and cats. In some instances, however, coronaviruses can jump to humans (i.e., spillover event). Of the seven known coronaviruses that have stricken humans, four cause only mild to moderate disease. However, three of the coronaviruses can cause more serious, even fatal, disease. SARS coronavirus (SARS-CoV) emerged in November 2002 and caused sever acute respiratory syndrome (SARS). That virus disappeared by 2004. Middle East respiratory syndrome (MERS) is caused by the MERS coronavirus (MERS-CoV). MERS was first identified in September 2012 after emerging from a camel reservoir. The third novel coronavirus to emerge in this century is called SARS-CoV-2. It causes coronavirus disease 2019 (i.e., COVID-19) which first emerged in China in December 2019, and was declared a global pandemic by the World Health Organization on Mar. 11, 2020.

As used herein, infection by the coronavirus SARS-CoV-2 causes coronavirus disease 2019, i.e., COVID-19. The term “coronavirus” or “SARS-CoV-2” encompassas wild-type virus and any mutants or variants thereof which results in viral infectious diseases including COVID-19. Variants include but are not limited to the “UK variant” known as 20I/501Y.V1, VOC 202012/01, or B. 1.1.7 that may be associated with an increased risk of death compared with other variants; the South Africa variant known as 20H/501Y.V2 or B. 1.351; and the Brazilian variant known as P. 1

As used herein, the term “disease” or “condition” refers to a variety of health abnormalities and/or conditions in a mammal including a human as generally understood, for example, in the medical profession, and further as described herein.

As used herein, “pre-existing condition” refers to one or more diseases or medical conditions including, but not limited to, diabetes, heart disease, chronic kidney disease, obesity, liver disease, hypertension, people 65 years or older, chronic lung disease, asthma, or immunocompromised individuals, that may predispose or render such individuals at higher risk for severe illness from coronavirus infection including infection by SARS-CoV-2.

As used herein, “coronavirus complication(s)” or “complication(s)” refers to one or more serious conditions tht can arise during or after COVID-19 infection and/or illness including, but not limited to, acute respiratory failure, pneumonia, acute respiratory distress syndrome (ARDS), lung or pulmonary fibrosis, scleroderma, acute liver injury, acute cardiac injury, secondary infection, acute kidney injury, septic shock, disseminated intravascular coagulation, and rhabdomylosis.

As used herein, the term “branched” refers to a group containing from 1 to 24 backbone atoms wherein the backbone chain of the group contains one or more subordinate branches from the main chain. Preferred branched groups herein contain from 1 to 12 backbone atoms. Examples of branched groups include, but are not limited to, isobutyl, t-butyl, isopropyl, --CH₂CH₂CH(CH₃)CH₂CH₃, —CH₂CH(CH₂CH₃)CH₂CH₃, —CH₂CH₂C(CH₃)₂CH₃, —CH₂CH₂C(CH₃)₃ and the like.

The term “unbranched” as used herein refers to a group containing from 1 to 24 backbone atoms wherein the backbone chain of the group extends in a direct line. Preferred unbranched groups herein contain from 1 to 12 backbone atoms.

The term “cyclic” or “cyclo” as used herein alone or in combination refers to a group having one or more closed rings, whether unsaturated or saturated, possessing rings of from 3 to 12 backbone atoms, preferably 3 to 7 backbone atoms.

The term “lower” as used herein refers to a group with 1 to 6 backbone atoms.

The term “saturated” as used herein refers to a group where all available valence bonds of the backbone atoms are attached to other atoms. Representative examples of saturated groups include, but are not limited to, butyl, cyclohexyl, piperidine and the like.

The term “unsaturated” as used herein refers to a group where at least one available valence bond of two adjacent backbone atoms is not attached to other atoms. Representative examples of unsaturated groups include, but are not limited to, —CH₂CH₂CH═CH₂, phenyl, pyrrole and the like.

The term “aliphatic” as used herein refers to an unbranched, branched or cyclic hydrocarbon group, which may be substituted or unsubstituted, and which may be saturated or unsaturated, but which is not aromatic. The term aliphatic further includes aliphatic groups, which comprise oxygen, nitrogen, sulfur or phosphorous atoms replacing one or more carbons of the hydrocarbon backbone.

The term “aromatic” as used herein refers to an unsaturated cyclic hydrocarbon group which may be substituted or unsubstituted having 4n+2 delocalized π(pi) electrons. The term aromatic further includes aromatic groups, which comprise a nitrogen atom replacing one or more carbons of the hydrocarbon backbone. Examples of aromatic groups include, but are not limited to, phenyl, naphthyl, thienyl, furanyl, pyridinyl, (is)oxazolyl and the like.

The term “substituted” as used herein refers to a group having one or more hydrogens or other atoms removed from a carbon or suitable heteroatom and replaced with a further group. Preferred substituted groups herein are substituted with one to five, most preferably one to three substituents. An atom with two substituents is denoted with “di” whereas an atom with more than two substituents is denoted by “poly.” Representative examples of such substituents include, but are not limited to aliphatic groups, aromatic groups, alkyl, alkenyl, alkynyl, aryl, alkoxy, halo, aryloxy, carbonyl, acryl, cyano, amino, amide, nitro, phosphate-containing groups, sulfur-containing groups, hydroxyl, alkylcarbonyloxy, arylcarbonyloxy, alkoxycarbonyloxy, aryloxycarbonyloxy, alkylcarbonyl, arylcarbonyl, alkoxycarbonyl, aminocarbonyl, alkylaminocarbonyl, dialkylaminocarbonyl, alkylthiocarbonyl, acylamino, amidino, imino, alkylthio, arylthio, thiocarboxylate, alkylsulfinyl, trifluoromethyl, azido, heterocyclyl, alkylaryl, heteroaryl, semicarbazido, thiosemicarbazido, maleimido, oximino, imidate, cycloalkyl, cycloalkylcarbonyl, dialkylamino, arylcycloalkyl, arylcarbonyl, arylalkylcarbonyl, arylcycloalkylcarbonyl, arylphosphinyl, arylalkylphosphinyl, arylcycloalkylphosphinyl, arylphosphonyl, arylalkylphosphonyl, arylcycloalkylphosphonyl, arylsulfonyl, arylalkylsulfonyl, arylcycloalkylsulfonyl, combinations thereof, and substitutions thereto.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The terms “optionally substituted”, “optionally substituted alkyl”, “optionally substituted alkenyl”, “optionally substituted alkynyl”, “optionally substituted carbocyclic”, “optionally substituted aryl”, “optionally substituted heteroaryl”, “optionally substituted heterocyclic”, and any other optionally substituted group as used herein, refer to groups that are substituted or unsubstituted by independent replacement of one, two, or three or more of the hydrogen atoms thereon with substituents including, but not limited to: -F, -Cl, -Br, -I, -OH, protected hydroxy, alkoxy, oxo, thiooxo, -NO₂, -CN, -CF₃, -N₃, -NH₂, protected amino, -NH- alkyl, -NH-alkenyl, -NH-alkynyl, -NH-cycloalkyl, -NH-aryl, -NH-heteroaryl, -NH-heterocyclic, - dialkylamino, -diarylamino, -diheteroarylamino, -O-alkyl, -O-alkenyl, -O-alkynyl, -O-cycloalkyl, - O-aryl, -O-heteroaryl, -O-heterocyclic, -C(O)-alkyl, -C(O)-alkenyl, -C(O)-alkynyl, -C(O)- cycloalkyl, -C(O)-aryl, -C(O)-heteroaryl, -C(O)-heterocycloalkyl, -CONH₂, -CONH-alkyl, - CONH-alkenyl, -CONH-alkynyl, -CONH-cycloalkyl, -CONH-aryl, -CONH-heteroaryl, -CONH- heterocycloalkyl, -OCO₂-alkyl, -OCO₂-alkenyl, -OCO₂-alkynyl, -OCO₂-cycloalkyl, -OCO₂-aryl, - OCOz-heteroaryl, -OCO₂-heterocycloalkyl, -OCONH₂, -OCONH-alkyl, -OCONH-alkenyl, - OCONH-alkynyl, -OCONH-cycloalkyl, -OCONH-aryl, -OCONH-heteroaryl, -OCONH- heterocycloalkyl, -NHC(O)-alkyl, -NHC(O)-alkenyl, -NHC(O)-lkynyl, -NHC(O)-cycloalkyl, - NHC(O)-aryl, -NHC(O)-heteroaryl, -NHC(O)-heterocycloalkyl, -NHCO₂-alkyl, -NHCO₂-alkenyl, -NHCO₂-alkynyl, -NHCO₂-cycloalkyl, -NHCO₂-aryl, -NHCOz-heteroaryl, -NHCO₂- heterocycloalkyl, -NHC(O)NH₂, -NHC(O)NH-alkyl, -NHC(O)NH-alkenyl, -NHC(O)NH-alkenyl, -NHC(O)NH-cycloalkyl, -NHC(O)NH-aryl, -NHC(O)NH-heteroaryl, -NHC(O)NH- heterocycloalkyl, -NHC(S)NH₂, -NHC(S)NH-alkyl, -NHC(S)NH-alkenyl, -NHC(S)NH-alkynyl, - NHC(S)NH-cycloalkyl, -NHC(S)NH-aryl, -NHC(S)NH-heteroaryl, -NHC(S)NH- heterocycloalkyl, -NHC(NH)NH₂, -NHC(NH)NH-alkyl, -NHC(NH)NH-alkenyl, -NHC(NH)NH- alkenyl, -NHC(NH)NH-cycloalkyl, -NHC(NH)NH-aryl, -NHC(NH)NH-heteroaryl, - NHC(NH)NH-heterocycloalkyl, -NHC(NH)-alkyl, -NHC(NH)-alkenyl, -NHC(NH)-alkenyl, - NHC(NH)-cycloalkyl, -NHC(NH)-aryl, -NHC(NH)-heteroaryl, -NHC(NH)-heterocycloalkyl, - C(NH)NH-alkyl, -C(NH)NH-alkenyl, -C(NH)NH-alkynyl, -C(NH)NH-cycloalkyl, -C(NH)NH- aryl, -C(NH)NH-heteroaryl, -C(NH)NH-heterocycloalkyl, -S(O)-alkyl, -S(O)-alkenyl, -S(O)- alkynyl, -S(O)-cycloalkyl, -S(O)-aryl, -S(O)-heteroaryl, -S(O)-heterocycloalkyl -SO₂NH₂, - SOzNH-alkyl, -SO₂NH-alkenyl, -SO₂NH-alkynyl, -SO₂NH-cycloalkyl, -SO₂NH-aryl, -SO₂NH- heteroaryl, -SO₂NH-heterocycloalkyl, -NHSO₂-alkyl, -NHSO₂-alkenyl, -NHSO₂-alkynyl, - NHSO₂-cycloalkyl, -NHSO₂-aryl, -NHSO₂-heteroaryl, -NHSO₂-heterocycloalkyl, -CH₂NH₂, -CH₂SO₂CH₃, -alkyl, -alkenyl, -alkynyl, -aryl, -arylalkyl, -heteroaryl, -heteroarylalkyl, -heterocycloalkyl, -cycloalkyl, -carbocyclic, -heterocyclic, polyalkoxyalkyl, polyalkoxy, methoxymethoxy, -methoxyethoxy, -SH, -S-alkyl, -S-alkenyl, -S-alkynyl, -S-cycloalkyl, -S-aryl, -S-heteroaryl, -S-heterocycloalkyl, or methylthiomethyl.

The term “unsubstituted” as used herein refers to a group that does not have any further groups attached thereto or substituted therefore.

The term “alkyl” as used herein, alone or in combination, refers to a branched or unbranched, saturated aliphatic group. The alkyl radical may be optionally substituted independently with one or more substituents described herein. Lower alkyl refers to alkyl groups of from one to six carbon atoms. Examples of lower alkyl groups include methyl, ethyl, propyl, isopropyl, butyl, s-butyl, t-butyl, isobutyl, pentyl, and the like. Higher alkyl refers to alkyl groups containing more than seven carbon atoms. A “Co” alkyl (as in “Co-Co-alkyl”) is a covalent bond. Exemplary alkyl groups are those of C₂₀ or below. In this application, alkyl refers to alkanyl, alkenyl, and alkynyl residues (and combinations thereof); it is intended to include vinyl, allyl, isoprenyl, and the like. Thus, when an alkyl residue having a specific number of carbons is named, all geometric isomers having that number of carbons are intended to be encompassed; thus, for example, either “butyl” or “C₄ alkyl” is meant to include n-butyl, sec-butyl, isobutyl, t-butyl, isobutenyl and but-2-ynyl groups; and for example, “propyl” or “C₃ alkyl” each include n-propyl, propenyl, and isopropyl. Representative examples of alkyl groups include, but are not limited to, methyl, ethyl, n-propyl, isopropyl, n-butyl, isobutyl, sec-butyl, tert-butyl, octyl, decyl, tetradecyl, hexadecyl, eicosyl, tetracosyl and the like. The terms “alkyl” or “alk” as used herein refer to a saturated linear or branched-chain monovalent hydrocarbon radical of one to twelve carbon atoms (C₁-C₁₂), wherein the alkyl radical may be optionally substituted independently with one or more substituents described below. In another embodiment, an alkyl radical is one to eight carbon atoms (C₁-C₈), or one to six carbon atoms (C₁-C₆). Examples of alkyl groups include, but are not limited to, methyl (Me, —CH₃), ethyl (Et, —CH₂CH₃), 1-propyl (n-Pr, n-propyl, --CH₂CH₂CH₃), 2-propyl (i-Pr, i-propyl, --CH(CH₃)₂), 1-butyl (n-Bu, n-butyl, --CH₂CH₂CH₂CH₃), 2-methyl-1-propyl (1-Bu, i-butyl, --CH₂CH(CH₃)₂), 2-butyl (s-Bu, s-butyl, --CH(CH₃)CH₂CH₃), 2-methyl-2-propyl (t-Bu, t-butyl, --C(CH3)₃), 1-pentyl (n-pentyl, --CH₂CH₂CH₂CH₂CH₃), 2-pentyl (-CH(CH₃)CH₂CH₂CH₃), 3-pentyl (--CH(CH₂CH₃)₂), 2-methyl-2-butyl (--C(CH₃)₂CH₂CH₃), 3-methyl-2-butyl (--CH(CH₃)CH(CH₃)z), 3-methyl-1-butyl (--CH₂CH₂CH(CH₃)₂), 2-methyl-1-butyl (-CH₂CH(CH₃)CH₂CH₃), 1-hexyl (--CH₂CH₂CH₂CH₂CH₂CH₃), 2-hexyl (-CH(CH₃)CH₂CH₂CH₂CH₃), 3-hexyl (--CH(CH₂CH₃)(CH₂CH₂CH₃)₂), 2-methyl-2-pentyl (--C(CH₃)₂CH₂CH₂CH₃), 3-methyl-2-pentyl (--CH(CH₃)CH(CH₃)CH₂CH₃), 4-methyl-2-pentyl (-CH(CH₃)CH₂CH(CH₃)₂), 3-methyl-3-pentyl (--C(CH₃)(CH₂CH₃)₂), 2-methyl-3-pentyl (-CH(CH₂CH₃)CH(CH₃)₂), 2,3-dimethyl-2-butyl (--C(CH₃)₂CH(CH₃)₂), 3,3-dimethyl-2-butyl (--CH(CH₃)C(CH₃)₃, 1-heptyl, 1-octyl, and the like.

The terms “carbocycle”, “carbocyclyl”, “carbocyclic ring” and “cycloalkyl” refer to a monovalent non-aromatic, saturated or partially unsaturated ring having 3 to 12 carbon atoms (C₃-C₁₂) as a monocyclic ring or 7 to 12 carbon atoms as a bicyclic ring. The cycloalkyl radical may be optionally substituted independently with one or more substituents described herein. Bicyclic carbocycles having 7 to 12 atoms can be arranged, for example, as a bicyclo[4,5], [5,5], [5,6] or [6,6] system, and bicyclic carbocycles having 9 or 10 ring atoms can be arranged as a bicyclo[5,6] or [6,6] system, or as bridged systems such as bicyclo[2.2.1]heptane, bicyclo[2.2.2]octane and bicyclo[3.2.2]nonane. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, cyclododecyl, and the like.

The term “alkenyl” as used herein alone or in combination refers to a branched or unbranched, unsaturated aliphatic group containing at least one carbon-carbon double bond which may occur at any stable point along the chain. The alkenyl radical may be optionally substituted independently with one or more substituents described herein, and includes radicals having “cis” and “trans” orientations, or alternatively, “E” and “Z” orientations. Representative examples of alkenyl groups include, but are not limited to, ethenyl, E- and Z-pentenyl, decenyl and the like.

The term “alkynyl” as used herein alone or in combination refers to a branched or unbranched, unsaturated aliphatic group containing at least one carbon-carbon triple bond which may occur at any stable point along the chain. The alkynyl radical may be optionally substituted independently with one or more substituents described herein. Representative examples of alkynyl groups include, but are not limited to, ethynyl, propynyl, propargyl, butynyl, hexynyl, decynyl and the like.

The term “aryl” as used herein alone or in combination refers to a substituted or unsubstituted aromatic group, which may be optionally fused to other aromatic or non-aromatic cyclic groups. Aryl includes bicyclic radicals comprising an aromatic ring fused to a saturated, partially unsaturated ring, or aromatic carbocyclic ring. Typical aryl groups include, but are not limited to, radicals derived from benzene (phenyl), substituted benzenes, naphthalene, anthracene, biphenyl, indenyl, indanyl, 1,2-dihydronaphthalene, 1,2,3,4-tetrahydronaphthyl, and the like. Aryl groups are optionally substituted independently with one or more substituents described herein.

The terms “heteroaryl” and “heteroar-”, used alone or as part of a larger moiety, e.g., “heteroaralkyl”, or “heteroaralkoxy”, refer to groups having 5 to 18 ring atoms, preferably 5, 6, 7, 9, or 14 ring atoms; having 6, 10, or 14 (pi) electrons shared in a cyclic array; and having, in addition to carbon atoms, from one to five heteroatoms. The term “heteroatom” includes but is not limited to nitrogen, oxygen, or sulfur, and includes any oxidized form of nitrogen or sulfur, and any quaternized form of a basic nitrogen. A heteroaryl may be a single ring, or two or more fused rings. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted. Examples include, but are not limited to, pyridinylmethyl, pyrimidinylethyl and the like.

The term “alkoxy” as used herein alone or in combination refers to an alkyl, alkenyl or alkynyl group bound through a single terminal ether linkage. Examples of alkoxy groups include, but are not limited to, methoxy, ethoxy, n-propoxy, iso-propoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, 3-methylpentoxy, fluoromethoxy, difluoromethoxy, trifluoromethoxy, chloromethoxy, dichloromethoxy, and trichloromethoxy.

The term “aryloxy” as used herein alone or in combination refers to an aryl group bound through a single terminal ether linkage.

The terms “halogen”, “halo” and “hal” as used herein refer to monovalent atoms of fluorine, chlorine, bromine, iodine and astatine.

The term “hetero” or “heteroatom” as used herein combination refers to a group that includes one or more atoms of any element other than carbon or hydrogen. Representative examples of hetero groups include, but are not limited to, those groups that contain heteroatoms including, but not limited to, nitrogen, oxygen, sulfur and phosphorus.

The term “heterocycle” or “heterocyclyl” or “heterocyclic ring” or “heterocyclic” as used herein refers to a cyclic group containing one or more heteroatoms. The heterocyclic radical may be optionally substituted independently with one or more substituents described herein. Representative examples of heterocycles include, but are not limited to, pyridine, piperidine, pyrimidine, pyridazine, piperazine, pyrrole, pyrrolidinone, pyrrolidine, morpholine, thiomorpholine, indole, isoindole, imidazole, triazole, tetrazole, furan, benzofuran, dibenzofuran, thiophene, thiazole, benzothiazole, benzoxazole, benzothiophene, quinoline, isoquinoline, azapine, naphthopyran, furanobenzopyranone and the like.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, 2-azabicyclo[2.2.1]heptanyl, octahydroindolyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

The term “covalent inhibitor” means an inhibitor of a target protein that forms a chemical bond by the sharing of electrons between atoms especially between sulfur atoms on a protein and the beta-carbon of an alpha-beta-unsaturated system present in the inhibitor small molecule typically through the use of Michael addition. The term “electrophile” means a positively charged or neutral species having vacant orbitals that are attracted to an electron rich centre (termed nucleophile). Electrophile groups on a covalent inhibitor participate in a chemical reaction by accepting an electron pair in order to bond to a nucleophile. The term “Michael addition” means the nucleophilic addition of a carbanion or another nucleophile to an a,p-unsaturated carbonyl compound. The term “covalent inhibitor” also means an inhibitor of a target protein that forms a chemical bond by the nucleophilic displacement of a leaving group (e.g., chlorine) on a primary or secondary carbon such as alpha-chloroacetamide, (preferably an alpha-chlorofluoroacetamide group) of the inhibitor by a sulfur atom of of the protein, for example cysteine or methionine (Naoya Shindo et al., Nature Chemical Biology, Vol. 15, March 2019 pp250-258 and reference therein).

The term “substituent” means any group selected from H, F, Cl, Br, I, alkyl, alkenyl, alkynyl, carbocycle, aryl, heterocycle, heteroaryl, formyl, nitro, cyano, amino, amide, carboxylic acid, carboxylic ester, carboxyl amide, reverse carboxyl amide, halo, haloalkyl, haloalkoxy, hydroxy, oxo (valency rules permitting), lower alkanyl, lower alkenyl, lower alkynyl, alkoxy, optionally substituted cycloalkyl, optionally substituted heterocycloalkyl, optionally’ substituted aryl, optionally substituted heteroaryl, alkylaminoalkyl, dialkylaminoalkyl, carboxy, carboxy ester, —C(O)NR⁵R″ (where R⁵ is hydrogen or alkyl and R″ is hydrogen, alkyl, aryl, or heterocyclyl), —NR⁵C(O)R″ (where R⁵ is hydrogen or alkyl and R″ is alkyl, aryl, or heterocyclyl), amino, alkylamino, dialkylamino, and —NHS(O)₂R′ (where R′ is alkyl, aryl, or heteroaryl).

The term “carbonyl” or “carboxy” as used herein alone or in combination refers to a group that contains a carbon-oxygen double bond. Representative examples of groups which contain a carbonyl include, but are not limited to, aldehydes (i.e., formyls), ketones (i.e., acyls), carboxylic acids (i.e., carboxyls), amides (i.e., amidos), imides (i.e., imidos), esters, anhydrides and the like.

The term “carbamate” as used herein alone or in combination refers to an ester group represented by the general structure —NH(CO)O—. Carbamate esters may have alkyl or aryl groups substituted on the nitrogen, or the amide function.

The term “cyanate” “isocyanate”, “thiocyanate”, or “isothiocyanate” as used herein alone or in combination refers to an oxygen- or sulfur-carbon double bond carbon-nitrogen double bond. Representative examples of cyano groups include, but are not limited to, isocyanate, isothiocyanate and the like.

The term “cyano”, “cyanide”, “isocyanide”, “nitrile”, or “isonitrile” as used herein alone or in combination refers to a carbon-nitrogen triple bond.

The term “amino” as used herein alone or in combination refers to a group containing a backbone nitrogen atom. Representative examples of amino groups include, but are not limited to, alkylamino, dialkylamino, arylamino, diarylamino, alkylarylamino, alkylcarbonylamino, arylcarbonylamino, carbamoyl, ureido and the like.

The term “phosphate-containing group” as used herein refers to a group containing at least one phosphorous atom in an oxidized state. Representative examples include, but are not limited to, phosphonic acids, phosphinic acids, phosphate esters, phosphinidenes, phosphinos, phosphinyls, phosphinylidenes, phosphos, phosphonos, phosphoranyls, phosphoranylidenes, phosphorosos and the like.

The term “sulfur-containing group” as used herein refers to a group containing a sulfur atom. Representative examples include, but are not limited to, sulfhydryls, sulfenos, sulfinos, sulfinyls, sulfos, sulfonyls, thios, thioxos and the like.

The term “optional” or “optionally” as used herein means that the subsequently described event or circumstance may or may not occur, and that the description includes instances where said event or circumstance occurs and instances where it does not. For example, the phrase “optionally substituted alkyl” means that the alkyl group may or may not be substituted and that the description includes both unsubstituted alkyl and substituted alkyl.

The term “targeting agent” as used herein means any compound of the invention or moiety attached to a compound of the invention allowing an increase in concentration of the compound at a site of treatment, for example, a tumor site. Exemplary targeting agents include but are not limited to carbohydrates, peptides, vitamins, and antibodies.

As used herein, the term “multi-target inhibitor” or “multi-target agent” refers to a single molecule having the capacity to interact with BET protein (BRD4 and/or BRD2) and at least one other protein target including but not limited to mTOR in vitro or in vivo including the capacity to inhibit the activity or normal function of said targets, e.g., to inhibit binding and/or enzymatic activity of BRD2/4 and mTOR.

As used herein, the term “dual inhibitor” refers to a single molecule that interacts with and/or inhibits the activity or normal function of two different target proteins, for example, BRD2/4 and PI3K or BRD2/4 and mTOR in vivo or in vitro.

The term “effective amount” or “effective concentration” when used in reference to a compound, product, or composition as provided herein, means a sufficient amount of the compound, product or composition to provide the desired pharmaceutical or therapeutic result. The exact amount required will vary depending on the particular compound, product or composition used, its mode of administration and the like. Thus, it is not always possible to specify an exact “effective amount.” However, an appropriate effective amount may be determined by one of ordinary skill in the art informed by the instant disclosure using only routine experimentation.

The term “hydrolyzable” as used herein refers to whether the group is capable of or prone to hydrolysis (i.e., splitting of the molecule or group into two or more new molecules or groups).

The term “pharmaceutically acceptable salt” of a compound of the instant invention (e.g., Formula I) is one which is the acid addition salt of a basic compound of the invention with an inorganic or organic acid which affords a physiologically acceptable anion or which is the salt formed by an acidic compound of the invention with a base which affords a physiologically acceptable cation.

The term “prodrug” or “procompound” as used in this application refers to a precursor or derivative form of a compound of the invention that may be less cytotoxic to cells compared to the parent compound or drug and is capable of being enzymatically or hydrolytically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, compounds of the invention and chemotherapeutic agents such as described above.

The term “conjugate” as used herein refers to a compound that has been formed by the joining of two or more compounds via either a covalent or non-covalent bond.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

A “metabolite” is a product of a compound or salt thereof produced by metabolism in the body. Metabolites of a compound may be identified using routine techniques known in the art, and their activities determined using tests such as those described herein. Such products may result, for example, from oxidation, reduction, hydrolysis, amidation, deamidation, esterification, deesterification, enzymatic cleavage, and the like, of an administered compound. Accordingly, the invention includes metabolites of compounds of the invention produced in vivo in a mammal including a human or produced in vitro.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If a compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If a compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

As used herein, the terms “treatment”, “treat”, and “treating” refer to preventing, reversing, alleviating, delaying the onset of, or inhibiting the progress of a disease or disorder including coronavirus infection and COVID-19 illness, or one or more symptoms or complications thereof, as may be described herein. In some embodiments, treatment may be administered before or after one or more symptoms have developed. In other embodiments, treatment may be administered in the absence of symptoms. For example, treatment may be administered to a susceptible individual or individual at high risk of infection prior to the onset of symptoms (i.e., in light of family history, medical history, pre-existing condition, symptoms, and genetic or other susceptibility factors). Treatment may also be continued after symptoms have resolved, for example, to prevent or delay their recurrence.

A “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include, but are not limited to, phenylsulfonylethyl, cyanoethyl, 2-(trimethylsilyl) ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The terms “compound of this invention,” and “compounds of the present invention” include compounds disclosed herein including but not limited to those of Formula I and stereoisomers, geometric isomers, tautomers, solvates, metabolites, and pharmaceutically acceptable salts, prodrugs, and conjugates thereof.

The term “TP scaffold” or “Thienopyranone scaffold” refers to a compound of general Formula I where M of the 5-membered ring is S.

The term “Furanopyranone scaffold” refers to a compound of Formula I where M of the 5-membered ring is O.

As used herein, the term “PI3K inhibiting” as applied to a compound of the invention means that a compound inhibits the normal or wild-type function of PI3K, i.e., enzymatic activity, in vivo and/or in vitro (e.g., PI3Kα, PI3Kβ, PI3Ky, PI3Kδ) with an IC₅₀ value of less than or equal to 50 µM in an appropriate in vitro assay.

As used herein, the term “Bromodomain inhibiting” as applied to a compound of the invention means that a compound inhibits the normal or wild-type function of a Bromodomain protein, in vivo and/or in vitro (e.g., BRD4) with an IC₅₀ value of less than or equal to 50 µM in an appropriate in vitro assay.

### B. Compounds

In one aspect, the present invention relates in part to single molecule, multitargeting compounds of Formula I and their use in therapeutic methods to treat and/or prevent viral infection by coronaviruses such as SARS coronavirus (SARS-CoV), MERS-CoV, and SARS-CoV-2 which casuses COVID-19 illness, by mechanisms incluing, but not limited to, inhibiting BET proteins such as but not limited to BRD2 and/or BRD4 and at least one other protein kinase such as, but not limited to, mTOR:

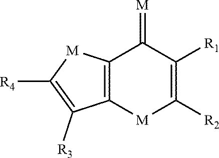

wherein M is independently oxygen (O) or sulfur (S);
R1 is selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from R1, morpholine, thiomorpholine, or piperazine;
R3 is selected from R1; and
R4 is selected from Rl.
R1-R4 of Formula I may independently contain varying amounts of isotopic substitution.

The present invention also relates to single molecule, multitargeting compounds of Formula II or a pharmaceutically acceptable salt thereof, and their use in therapeutic methods to treat and/or prevent viral infection by coronaviruses such as SARS coronavirus (SARS-CoV), MERS-CoV, and SARS-CoV-2 which casuses COVID-19 illness, by mechanisms incluing, but not limited to, inhibiting BRD2 and/or BRD4 and at least one protein kinase such as, but not limited to, mTOR:

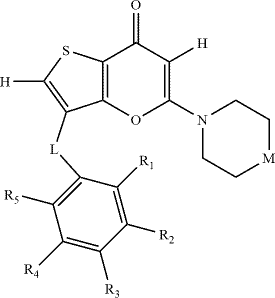

wherein M is independently oxygen (O) or sulfur (S) or N-R₁;
L is null or acetylenic;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from R1,
R3 is selected from R1;
R4 is selected from R1;
R5 is selected from R1; and
where R1-R5 may independently contain varying amounts of isotopic substitution.

Representative compounds of Formula II are shown in Table 1 below.

TABLE 1

Reprsentative compounds of Formula II

| Compound No. | Structure | Compound No. | Structure |
|---|---|---|---|
| 0 | 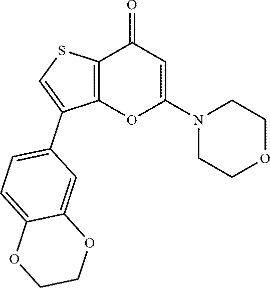 [SMILES: O=c1cc(N2CCOCC2)oc2c(-c3ccc4c(c3)OCCO4)csc12] | 1 | 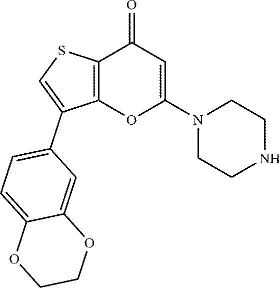 [SMILES: O=c1cc(N2CCNCC2)oc2c(-c3ccc4c(c3)OCCO4)csc12] |
| 2 | 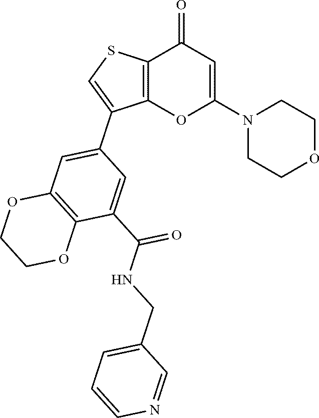 [SMILES: O=C(NCc1cccnc1)c1cc(-c2csc3c(=O)cc(N4CCOCC4)oc23)cc2c1OCCO2] | 3 | 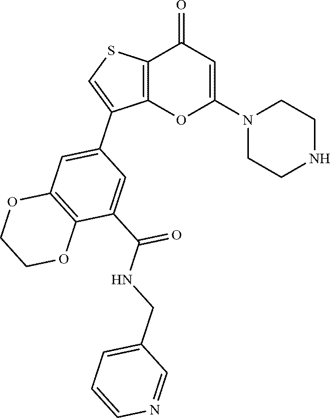 [SMILES: O=C(NCc1cccnc1)c1cc(-c2csc3c(=O)cc(N4CCNCC4)oc23)cc2c1OCCO2] |
| 4 | 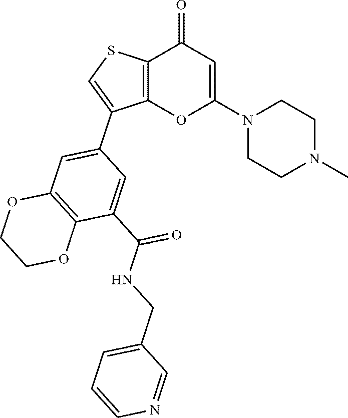 [SMILES: CN1CCN(c2cc(=O)c3scc(-c4cc5c(c(C(=O)NCc6cccnc6)c4)OCCO5)c3o2)CC1] | 5 | 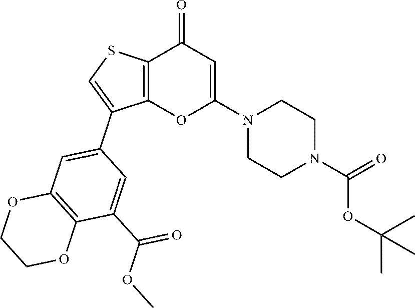 [SMILES: COC(=O)c1cc(-c2csc3c(=O)cc(N4CCN(C(=O)OC(C)(C)C)CC4)oc23)cc2c1OCCO2] |
| 5-1 | 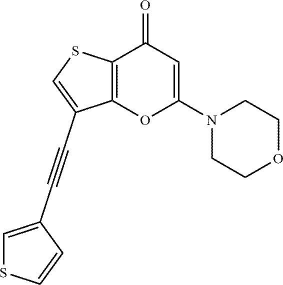 [SMILES: O=c1cc(N2CCOCC2)oc2c(C#Cc3ccsc3)csc12] | 5-2 | 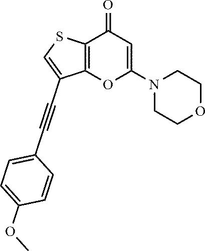 [SMILES: COc1ccc(C#Cc2csc3c(=O)cc(N4CCOCC4)oc23)cc1] |
| 5-3 | 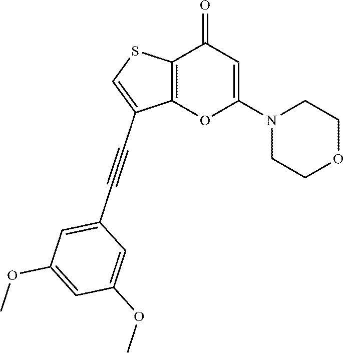 [SMILES: COc1cc(C#Cc2csc3c(=O)cc(N4CCOCC4)oc23)cc(OC)c1] | 5-4 | 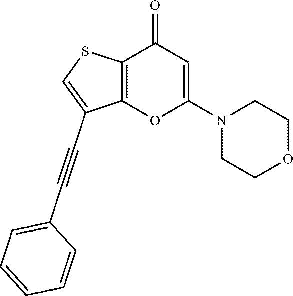 [SMILES: O=c1cc(N2CCOCC2)oc2c(C#Cc3ccccc3)csc12] |

The present invention also relates in part to single molecule, multitargeting compounds of Formula III or a pharmaceutically acceptable salt thereof, and their use in therapeutic methods to treat and/or prevent viral infection by coronaviruses such as SARS coronavirus (SARS-CoV), MERS-CoV, and SARS-CoV-2 which casuses COVID-19 illness, by mechanisms incluing, but not limited to, inhibiting BRD2 and/or BRD4 and at least one other protein kinase such as, but not limited to, mTOR:

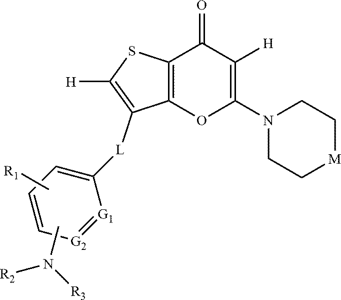

wherein M is independently oxygen (O) or sulfur (S) or N-R₁;
L is null, NH, or acetylenic;
G1 and G2 are independently selected from CH and N;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from hydrogen, methyl, or R3;
R3 is selected from

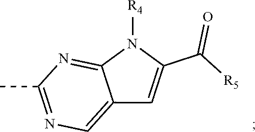

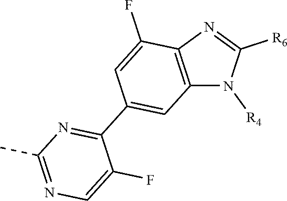

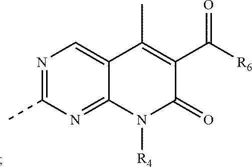

R4 is selected from a straight or branched or cyclic C1 to C6 aliphatic chain;
R5 is selected from hydroxyl, NH₂, NH(CH₃), N(CH₃)₂, NH-OH, NH-OCH₃; and
R6 is selected from a straight or branched or cyclic C1 to C6 aliphatic chain; and
where R1-R6 may independently contain varying amounts of isotopic substitution.

Representative compounds of Formula III are shown in Table 2 below.

TABLE 2

Representative compounds of Formula III

| Comp No. | Structure | Comp No | Structure |
|---|---|---|---|
| 6 | 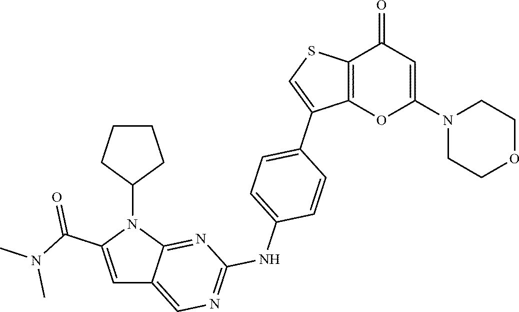 [SMILES: CN(C)C(=O)c1cc2cnc(Nc3ccc(-c4csc5c(=O)cc(N6CCOCC6)oc45)cc3)nc2n1C1CCCC1] | 7 | 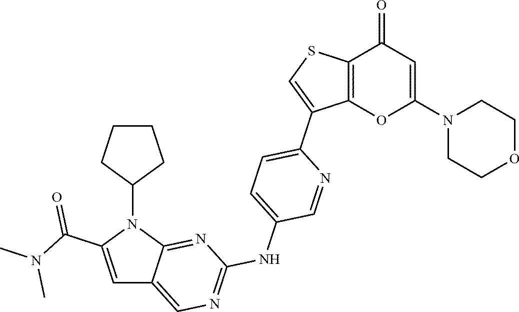 [SMILES: CN(C)C(=O)c1cc2cnc(Nc3ccc(-c4csc5c(=O)cc(N6CCOCC6)oc45)nc3)nc2n1C1CCCC1] |
| 8 | 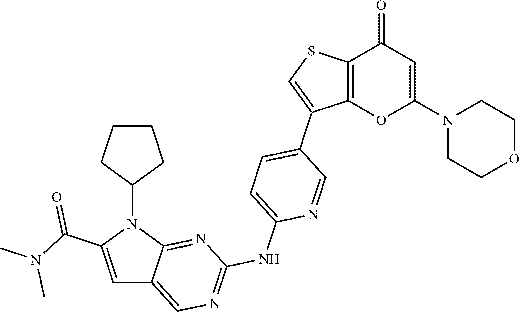 [SMILES: CN(C)C(=O)c1cc2cnc(Nc3ccc(-c4csc5c(=O)cc(N6CCOCC6)oc45)cn3)nc2n1C1CCCC1] | 9 | 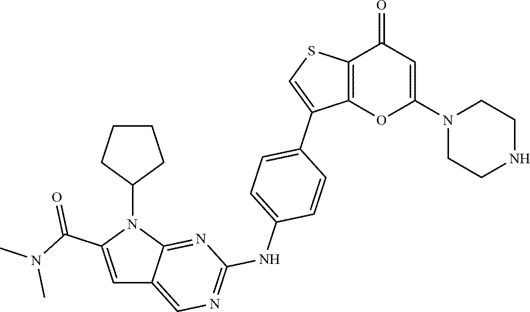 [SMILES: CN(C)C(=O)c1cc2cnc(Nc3ccc(-c4csc5c(=O)cc(N6CCNCC6)oc45)cc3)nc2n1C1CCCC1] |
| 10 | 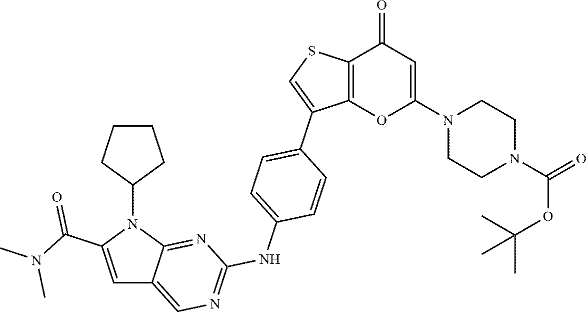 [SMILES: CN(C)C(=O)c1cc2cnc(Nc3ccc(-c4csc5c(=O)cc(N6CCN(C(=O)OC(C)(C)C)CC6)oc45)cc3)nc2n1C1CCCC1] | 11 | 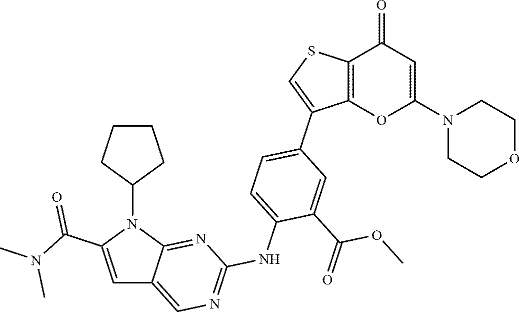 [SMILES: COC(=O)c1cc(-c2csc3c(=O)cc(N4CCOCC4)oc23)ccc1Nc1ncc2cc(C(=O)N(C)C)n(C3CCCC3)c2n1] |
| 12 | 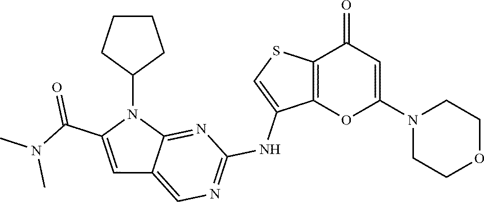 [SMILES: CN(C)C(=O)c1cc2cnc(Nc3csc4c(=O)cc(N5CCOCC5)oc34)nc2n1C1CCCC1] | 13 | 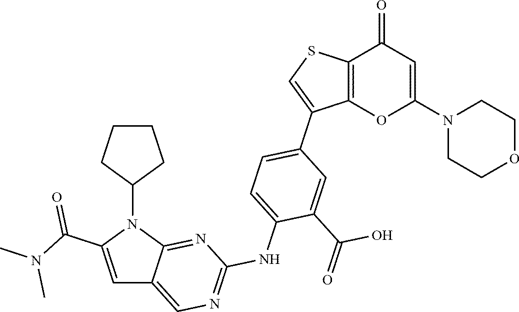 [SMILES: CN(C)C(=O)c1cc2cnc(Nc3ccc(-c4csc5c(=O)cc(N6CCOCC6)oc45)cc3C(=O)O)nc2n1C1CCCC1] |
| 14 | 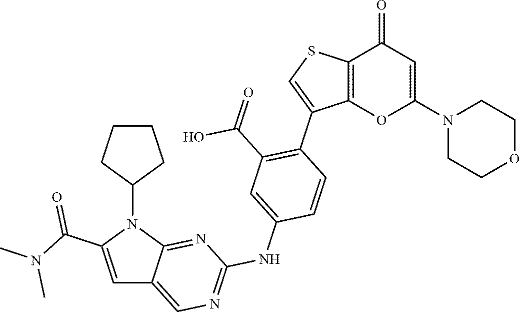 [SMILES: CN(C)C(=O)c1cc2cnc(Nc3ccc(-c4csc5c(=O)cc(N6CCOCC6)oc45)c(C(=O)O)c3)nc2n1C1CCCC1] | 15 | 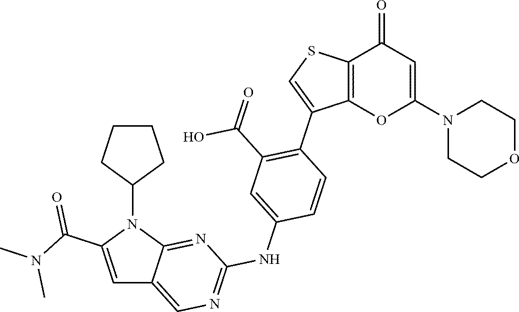 [SMILES: CN(C)C(=O)c1cc2cnc(Nc3ccc(-c4csc5c(=O)cc(N6CCOCC6)oc45)c(C(=O)O)c3)nc2n1C1CCCC1] |
| 16 | 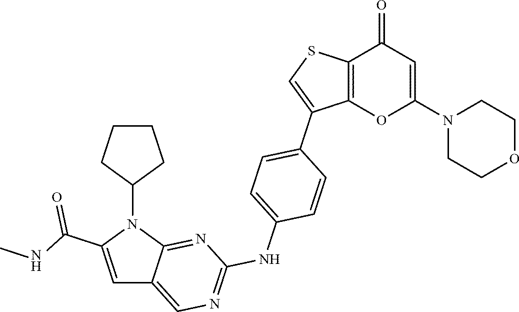 [SMILES: CNC(=O)c1cc2cnc(Nc3ccc(-c4csc5c(=O)cc(N6CCOCC6)oc45)cc3)nc2n1C1CCCC1] | 17 | 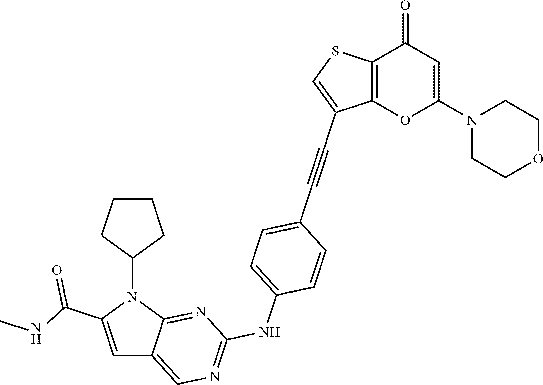 [SMILES: CNC(=O)c1cc2cnc(Nc3ccc(C#Cc4csc5c(=O)cc(N6CCOCC6)oc45)cc3)nc2n1C1CCCC1] |
| 18 | 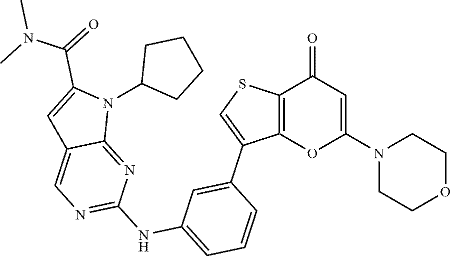 [SMILES: CN(C)C(=O)c1cc2cnc(Nc3cccc(-c4csc5c(=O)cc(N6CCOCC6)oc45)c3)nc2n1C1CCCC1] | 19 | 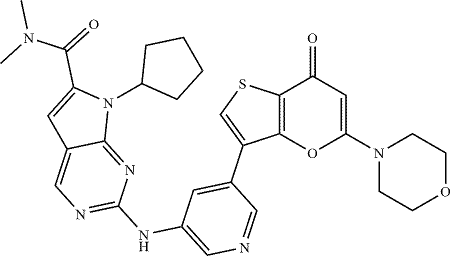 [SMILES: CN(C)C(=O)c1cc2cnc(Nc3cncc(-c4csc5c(=O)cc(N6CCOCC6)oc45)c3)nc2n1C1CCCC1] |
| 20 | 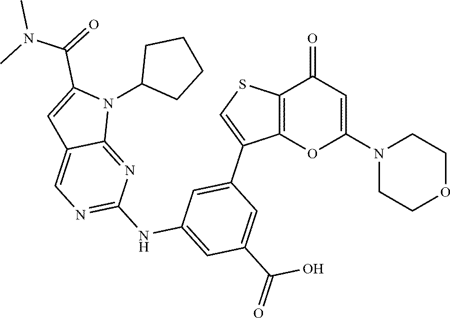 [SMILES: CN(C)C(=O)c1cc2cnc(Nc3cc(C(=O)O)cc(-c4csc5c(=O)cc(N6CCOCC6)oc45)c3)nc2n1C1CCCC1] | 21 | 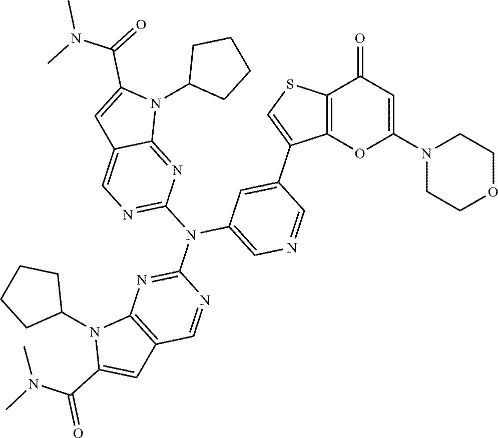 [SMILES: CN(C)C(=O)c1cc2cnc(N(c3cncc(-c4csc5c(=O)cc(N6CCOCC6)oc45)c3)c3ncc4cc(C(=O)N(C)C)n(C5CCCC5)c4n3)nc2n1C1CCCC1] |
| 22 | 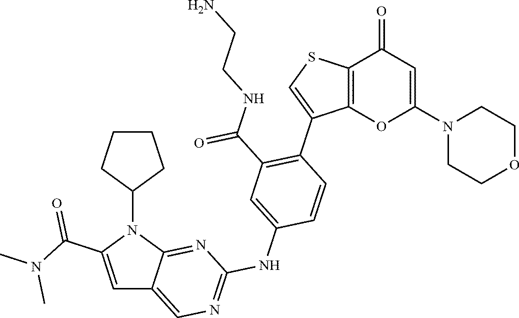 [SMILES: CN(C)C(=O)c1cc2cnc(Nc3ccc(-c4csc5c(=O)cc(N6CCOCC6)oc45)c(C(=O)NCCN)c3)nc2n1C1CCCC1] | 23 | 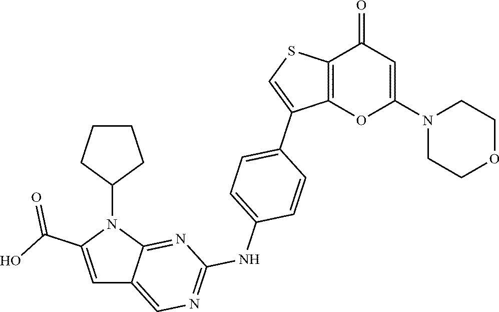 [SMILES: O=C(O)c1cc2cnc(Nc3ccc(-c4csc5c(=O)cc(N6CCOCC6)oc45)cc3)nc2n1C1CCCC1] |
| 24 | 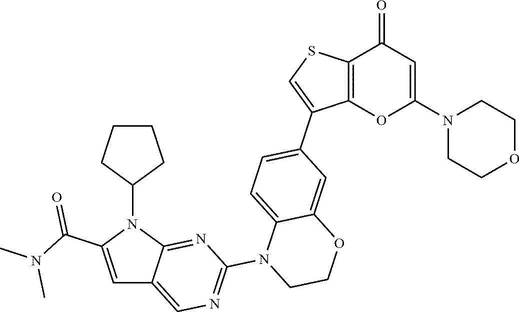 [SMILES: CN(C)C(=O)c1cc2cnc(N3CCOc4cc(-c5csc6c(=O)cc(N7CCOCC7)oc56)ccc43)nc2n1C1CCCC1] | 25 | 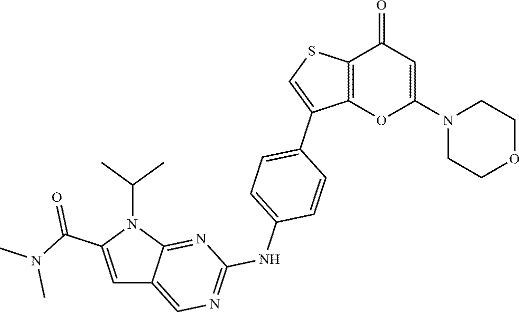 [SMILES: CC(C)n1c(C(=O)N(C)C)cc2cnc(Nc3ccc(-c4csc5c(=O)cc(N6CCOCC6)oc45)cc3)nc21] |
| 25-1 | 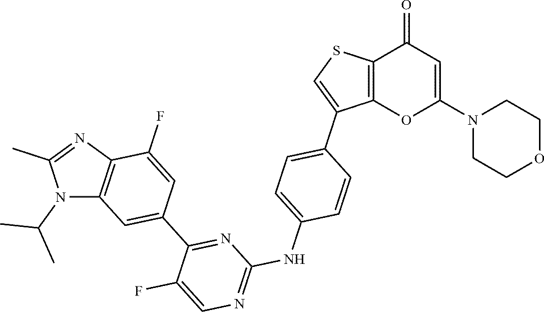 [SMILES: Cc1nc2c(F)cc(-c3nc(Nc4ccc(-c5csc6c(=O)cc(N7CCOCC7)oc56)cc4)ncc3F)cc2n1C(C)C] | 25-2 | 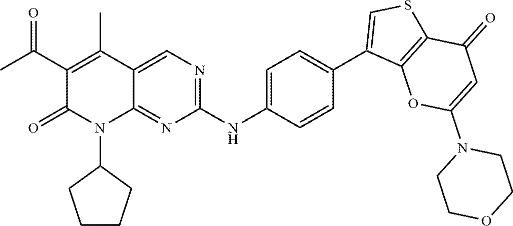 [SMILES: CC(=O)c1c(C)c2cnc(Nc3ccc(-c4csc5c(=O)cc(N6CCOCC6)oc45)cc3)nc2n(C2CCCC2)c1=O] |
| 25-3 | 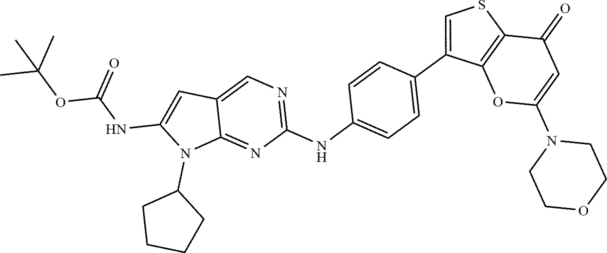 [SMILES: CC(C)(C)OC(=O)Nc1cc2cnc(Nc3ccc(-c4csc5c(=O)cc(N6CCOCC6)oc45)cc3)nc2n1C1CCCC1] | | |

The present invention also relates in part to single molecule, multitargeting compounds of Formula IV and their use in therapeutic methods to treat and/or prevent viral infection by coronaviruses such as SARS coronavirus (SARS-CoV), MERS-CoV, and SARS-CoV-2 which casuses COVID-19 illness, by mechanisms incluing, but not limited to, inhibiting BRD2 and/or BRD4 and at least one other protein kinase such as, but not limited to, mTOR:

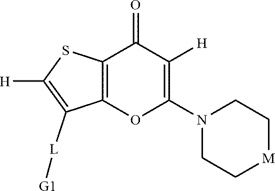

wherein M is independently oxygen (O) or sulfur (S) or N-R₁;
L is null or acetylenic;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
G1 is selected from

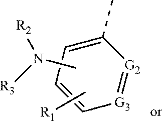

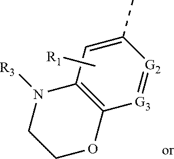

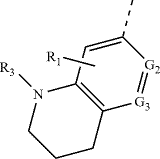

G2 and G3 are independently selected from CR1 and N;
R2 is selected from hydrogen, methyl, ethyl, 2-hydroxyethyl, 2-methoxyethyl, or R3;
R3 is selected from —S(O)(O)—R4 or —C(O)—R4;
R4 is selected from

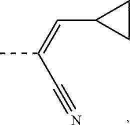

C2 to C10 alkyl, carbocyclic, alkenyl, alkynyl chain or cycloalkenyl; and
where R1-R4 may independently contain varying amounts of isotopic substitution.

Representative compounds of Formula IV are shown in Table 3 below.

TABLE 3

Representative compounds of Formula IV

| Comp No. | Structure | Comp No. | Structure |
|---|---|---|---|
| 26 | 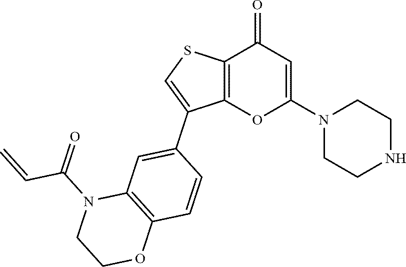 [SMILES: C=CC(=O)N1CCOc2ccc(-c3csc4c(=O)cc(N5CCNCC5)oc34)cc21] | 27 | 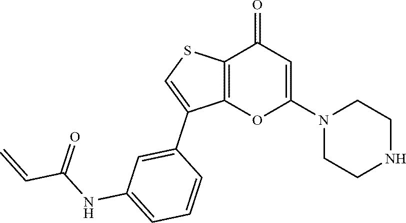 [SMILES: C=CC(=O)Nc1cccc(-c2csc3c(=O)cc(N4CCNCC4)oc23)c1] |
| 28 | 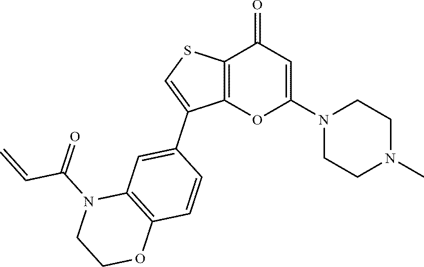 [SMILES: C=CC(=O)N1CCOc2ccc(-c3csc4c(=O)cc(N5CCN(C)CC5)oc34)cc21] | 29 | 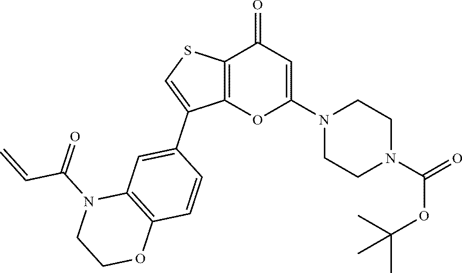 [SMILES: C=CC(=O)N1CCOc2ccc(-c3csc4c(=O)cc(N5CCN(C(=O)OC(C)(C)C)CC5)oc34)cc21] |
| 30 | 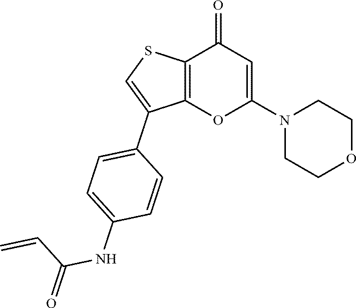 [SMILES: C=CC(=O)Nc1ccc(-c2csc3c(=O)cc(N4CCOCC4)oc23)cc1] | 31 | 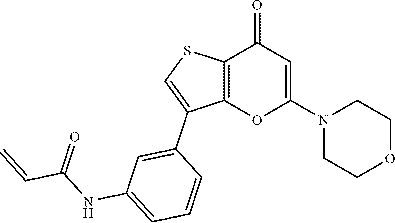 [SMILES: C=CC(=O)Nc1cccc(-c2csc3c(=O)cc(N4CCOCC4)oc23)c1] |
| 32 | 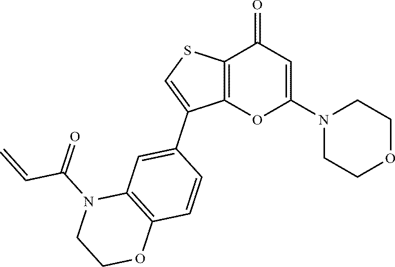 [SMILES: C=CC(=O)N1CCOc2ccc(-c3csc4c(=O)cc(N5CCOCC5)oc34)cc21] | 33 | 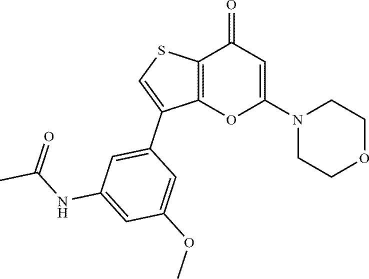 [SMILES: COc1cc(NC(C)=O)cc(-c2csc3c(=O)cc(N4CCOCC4)oc23)c1] |
| 34 | 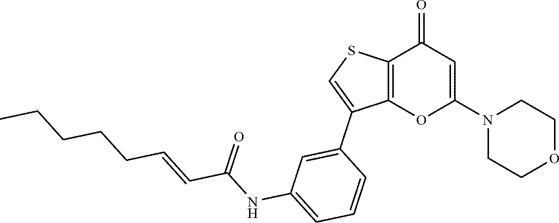 [SMILES: CCCCC/C=C/C(=O)Nc1cccc(-c2csc3c(=O)cc(N4CCOCC4)oc23)c1] | 35 | 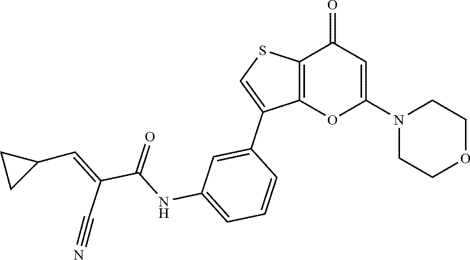 [SMILES: N#C/C(=C\C1CC1)C(=O)Nc1cccc(-c2csc3c(=O)cc(N4CCOCC4)oc23)c1] |
| 36 | 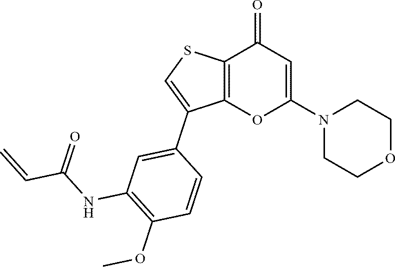 [SMILES: C=CC(=O)Nc1cc(-c2csc3c(=O)cc(N4CCOCC4)oc23)ccc1OC] | 37 | 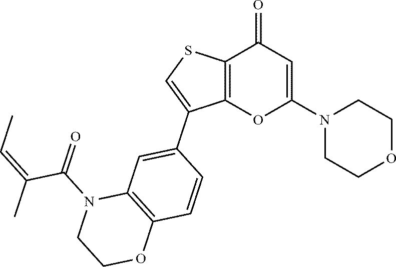 [SMILES: C/C=C(/C)C(=O)N1CCOc2ccc(-c3csc4c(=O)cc(N5CCOCC5)oc34)cc21] |
| 38 | 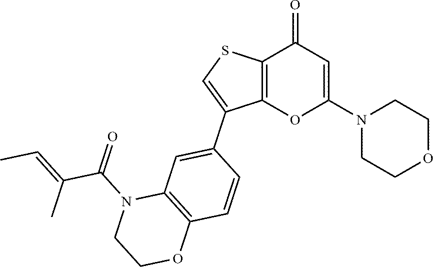 [SMILES: C/C=C(\C)C(=O)N1CCOc2ccc(-c3csc4c(=O)cc(N5CCOCC5)oc34)cc21] | 39 | 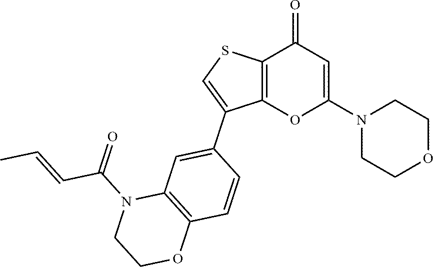 [SMILES: C/C=C/C(=O)N1CCOc2ccc(-c3csc4c(=O)cc(N5CCOCC5)oc34)cc21] |
| 40 | 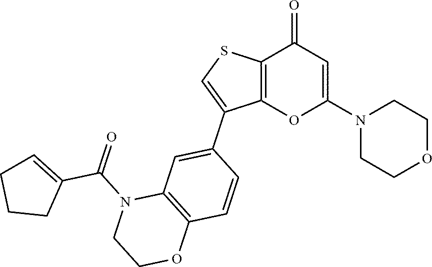 [SMILES: O=C(C1=CCCC1)N1CCOc2ccc(-c3csc4c(=O)cc(N5CCOCC5)oc34)cc21] | 41 | 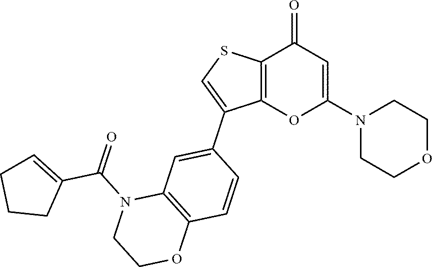 [SMILES: O=C(C1=CCCC1)N1CCOc2ccc(-c3csc4c(=O)cc(N5CCOCC5)oc34)cc21] |
| 42 | 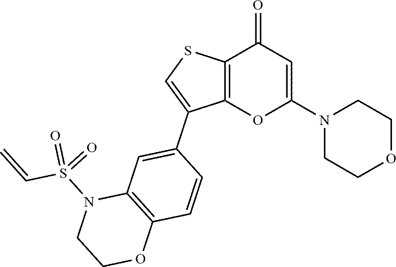 [SMILES: C=CS(=O)(=O)N1CCOc2ccc(-c3csc4c(=O)cc(N5CCOCC5)oc34)cc21] | 43 | 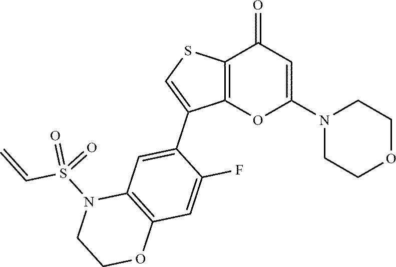 [SMILES: C=CS(=O)(=O)N1CCOc2cc(F)c(-c3csc4c(=O)cc(N5CCOCC5)oc34)cc21] |
| 44 | 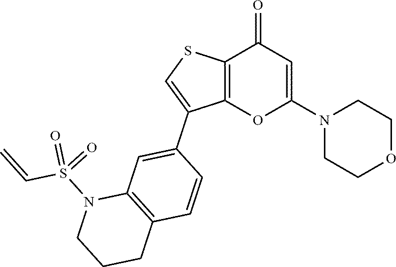 [SMILES: C=CS(=O)(=O)N1CCCc2ccc(-c3csc4c(=O)cc(N5CCOCC5)oc34)cc21] | 45 | 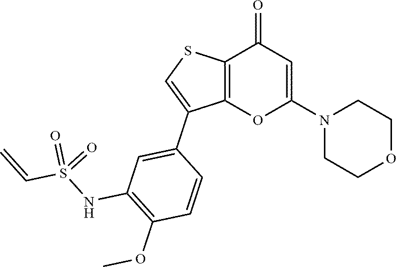 [SMILES: C=CS(=O)(=O)Nc1cc(-c2csc3c(=O)cc(N4CCOCC4)oc23)ccc1OC] |
| 46 | 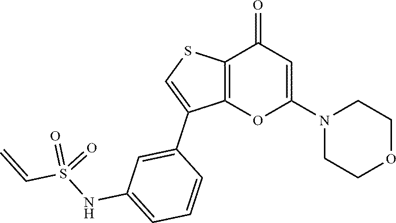 [SMILES: C=CS(=O)(=O)Nc1cccc(-c2csc3c(=O)cc(N4CCOCC4)oc23)c1] | 47 | 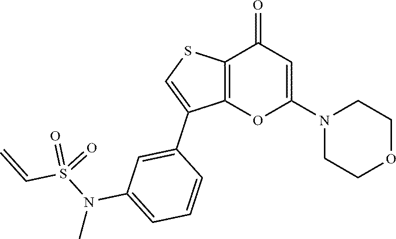 [SMILES: C=CS(=O)(=O)N(C)c1cccc(-c2csc3c(=O)cc(N4CCOCC4)oc23)c1] |
| 48 | 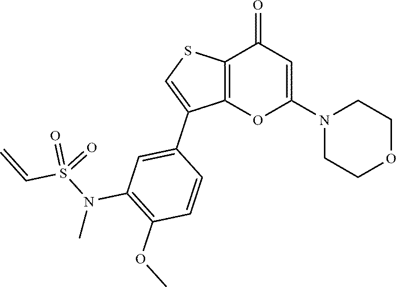 [SMILES: C=CS(=O)(=O)N(C)c1cc(-c2csc3c(=O)cc(N4CCOCC4)oc23)ccc1OC] | 49 | 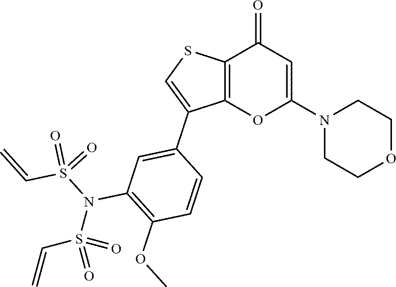 [SMILES: C=CS(=O)(=O)N(c1cc(-c2csc3c(=O)cc(N4CCOCC4)oc23)ccc1OC)S(=O)(=O)C=C] |
| 50 | 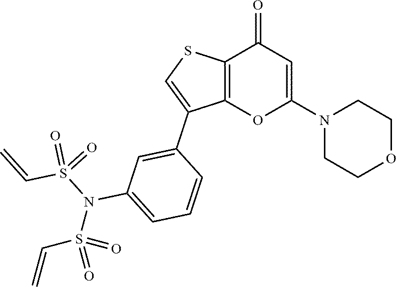 [SMILES: C=CS(=O)(=O)N(c1cccc(-c2csc3c(=O)cc(N4CCOCC4)oc23)c1)S(=O)(=O)C=C] | 51 | 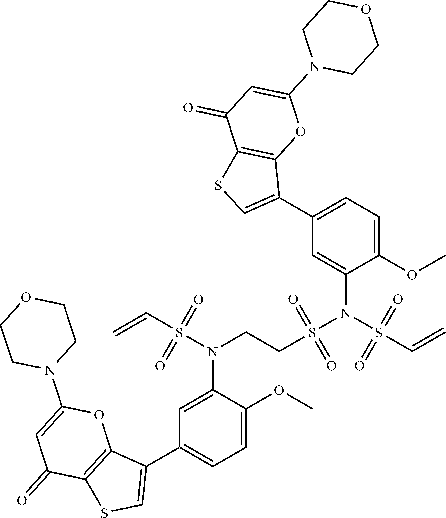 [SMILES: C=CS(=O)(=O)N(CCS(=O)(=O)N(c1cc(-c2csc3c(=O)cc(N4CCOCC4)oc23)ccc1OC)S(=O)(=O)C=C)c1cc(-c2csc3c(=O)cc(N4CCOCC4)oc23)ccc1OC] |

The present invention also relates in part to single molecule, multitargeting compounds of Formula V and their use in therapeutic methods to treat and/or prevent viral infection by coronaviruses such as SARS coronavirus (SARS-CoV), MERS-CoV, and SARS-CoV-2 which casuses COVID-19 illness, by mechanisms incluing, but not limited to, inhibiting BRD2 and/or BRD4 and at least one other protein kinase such as, but not limited to, mTOR:

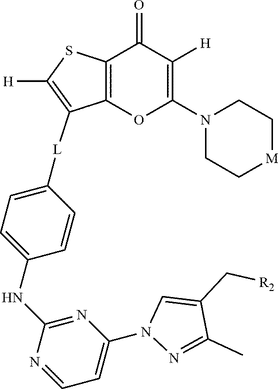

wherein M is independently oxygen (O) or sulfur (S) or N-R₁;
L is null or acetylenic;
R1 is independently selected from H, methyl, —C(O)OC(CH₃)₃;
R2 is selected from

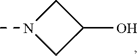

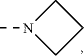

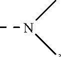

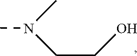

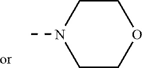

and;
where R1-R2 may independently contain varying amounts of isotopic substitution.

Representative compounds of Formula V are shown in Table 4 below.

TABLE 4

Representative compounds of Formula V

| Comp No. | Structure | Comp No. | Structure |
|---|---|---|---|
| 52 | 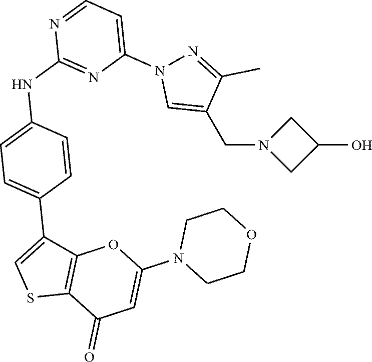 [SMILES: Cc1nn(-c2ccnc(Nc3ccc(-c4csc5c(=O)cc(N6CCOCC6)oc45)cc3)n2)cc1CN1CC(O)C1] | 53 | 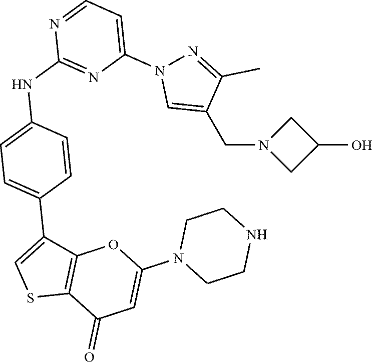 [SMILES: Cc1nn(-c2ccnc(Nc3ccc(-c4csc5c(=O)cc(N6CCNCC6)oc45)cc3)n2)cc1CN1CC(O)C1] |
| 54 | 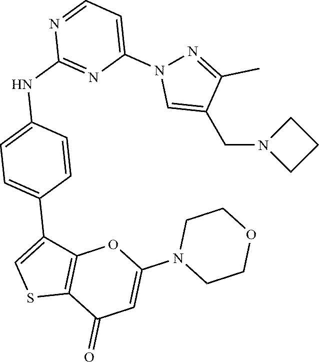 [SMILES: Cc1nn(-c2ccnc(Nc3ccc(-c4csc5c(=O)cc(N6CCOCC6)oc45)cc3)n2)cc1CN1CCC1] | 55 | 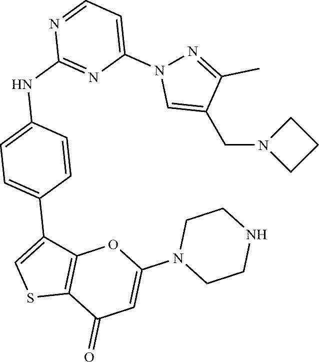 [SMILES: Cc1nn(-c2ccnc(Nc3ccc(-c4csc5c(=O)cc(N6CCNCC6)oc45)cc3)n2)cc1CN1CCC1] |
| 56 | 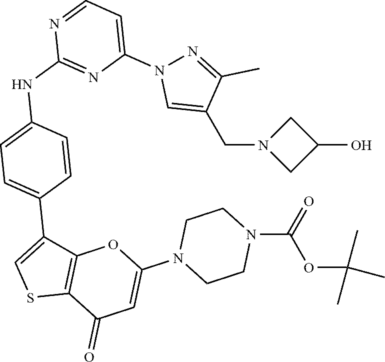 [SMILES: Cc1nn(-c2ccnc(Nc3ccc(-c4csc5c(=O)cc(N6CCN(C(=O)OC(C)(C)C)CC6)oc45)cc3)n2)cc1CN1CC(O)C1] | 57 | 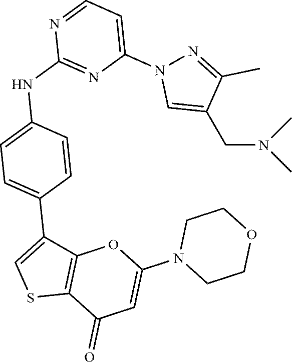 [SMILES: Cc1nn(-c2ccnc(Nc3ccc(-c4csc5c(=O)cc(N6CCOCC6)oc45)cc3)n2)cc1CN(C)C] |
| 58 | 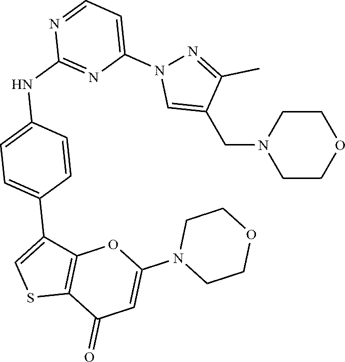 [SMILES: Cc1nn(-c2ccnc(Nc3ccc(-c4csc5c(=O)cc(N6CCOCC6)oc45)cc3)n2)cc1CN1CCOCC1] | 59 | 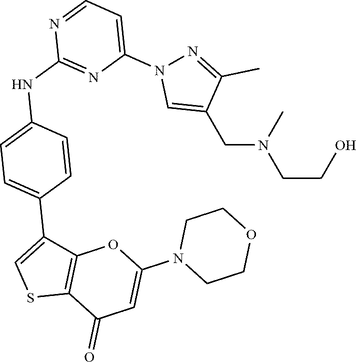 [SMILES: Cc1nn(-c2ccnc(Nc3ccc(-c4csc5c(=O)cc(N6CCOCC6)oc45)cc3)n2)cc1CN(C)CCO] |

In another aspect of the invention, a pharmaceutically acceptable salt of a compound of the invention is one which is the acid addition salt of a basic compound of Formula I with an inorganic or organic acid which affords a physiologically acceptable anion, or which is the salt formed by an acidic compound of Formula I with a base which affords a physiologically acceptable cation. Examples of such acids and bases are provided hereinbelow.

Another aspect of the invention relates to methods of using a pharmaceutical formulation comprising a compound of Formula I in association with a pharmaceutically acceptable carrier, diluent or excipient, a compound of Formula I (or a pharmaceutically acceptable salt thereof), as provided in any of the descriptions herein.

In addition, compounds (or salts thereof) of the present invention are useful as an active ingredient in the manufacture of a medicament for the treatment of diseases including, but not limited to, coronavirus infections, and/or for inhibiting BRD2/4 and at least one other protein kinase including but not limited to mTOR for the treatment of viral infectious diseases including but not limited to COVID-19

The present invention also provides a method for treating a disease in a human or other mammal including, but not limited to, COVID-19 and/or complications thereof by administering a therapeutically effective amount of a compound(s) of the invention including compound(s) or composition(s) of Formula I or conjugate or prodrug thereof having any of the definitions herein.

The present invention further provides a method for inhibiting BRD2/4 and at least one other protein kinase including but not limited to mTOR in a mammal including a human in need thereof by administering an effective amount of a compound of Formula I, or conjugate or prodrug thereof having any of the definitions herein.

Compounds of the invention may be co-administered with one or more other agents to treat coronavirus infections including but not limted to chloroquine, hydroxychloroquine, and Remdesivir.

It will be appreciated that certain compounds of Formula I (or salts, procompounds, conjugates, etc.) may exist in, and be isolated in, isomeric forms, including tautomeric forms, cis-or trans-isomers, as well as optically active, racemic, enantiomeric or diastereomeric forms. It is to be understood that the present invention encompasses a compound of Formula I for use in a method described herein in any of the tautomeric forms or as a mixture thereof; or as a mixture of diastereomers, as well as in the form of an individual diastereomer, and that the present invention encompasses a compound of Formula I as a mixture of enantiomers, as well as in the form of an individual enantiomer, any of which mixtures or form desirably possesses inhibitory properties against kinases including but not limited to PI3 kinase, it being well known in the art how to prepare or isolate particular forms and how to determine inhibitory properties against kinases by standard tests including those described herein below.

In addition, a compound of Formula I (or salt, procompound, conjugate thereof, etc.) may exhibit polymorphism or may form a solvate with water or an organic solvent. The present invention also encompasses any such polymorphic form, any solvate or any mixture thereof, for use in the methods described herein.

The methods of the invention include manufacturing and administering a pharmaceutically acceptable salt of a compound of Formula I. A basic compound of the invention possesses one or more functional groups sufficiently basic to react with any of a number of inorganic and organic acids affording a physiologically acceptable counterion to form a pharmaceutically acceptable salt. Acids commonly employed to form pharmaceutically acceptable acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromobenzenesulfonic acid, carbonic acid, succinic acid, citric acid, benzoic acid, acetic acid, and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycollate, tartrate, methanesulfonate, propanesulfonate, naphthalene-1-sulfonate, naphthalene-2-sulfonate, mandelate, and the like. Preferred pharmaceutically acceptable acid addition salts include those formed with mineral acids such as hydrochloric acid, hydrobromic acid and sulfuric acid.

### C.I. Synthesis of Compounds and Conjugates

Compounds of the invention may be prepared according to the examples provided herein as well as by processes known in the chemical arts and described, for example, in US Pat. 8,557,807 and references cited therein, as well as in G.A. Morales et al., J. Med. Chem. 2013, 56, 1922-1939, the entire contents of which are herein incorporated by reference. Starting materials and intermediates used to prepare a compound of the invention are either commercially available or can be readily prepared by one of ordinary skill in the art. Compounds and conjugates described herein and used in the therapeutic methods of the invention can be made, for example, by procedures disclosed in US Pat. 6,949,537; 7,662,977; 7,396,828; 8,557,807; and 9,505,780; and in US Pat. Applications 14/702816, and 15/297293, the entire contents of which are herein incorporated by reference. Thio compounds can be made from oxygen analogs as described in the art, for example by using Lawesson’s reagent as described in Morales et al., J. Med. Chem. 2013. Furan analogs of the thiophene-pyranone compounds (termed thienopyranones) can be made, for example, by the general schemes outlined below where the key intermediate “g” is prepared and utilized. Intermediate “g” is then further elaborated to the oxygen analog of “compound 6” as described in Morales et al., J. Med. Chem. 2013 (reference incorporated herein) which is designated below as compound “i”. Compound “i” can then be reacted via couplings with boronates to make the final substituted furanopyranones of the invention. Alternatively, the bromine atom in compound “i” can be converted to a boron derivative and then coupled with aryl or heteroaryl bromides or iodides to make furanopyranones of the invention.

A reaction scheme is shown below for preparing furanopyranones of the invention via the key furan intermediate “g” and subsequent conversion to compound “i” which is then further reacted to produce compounds of the invention:

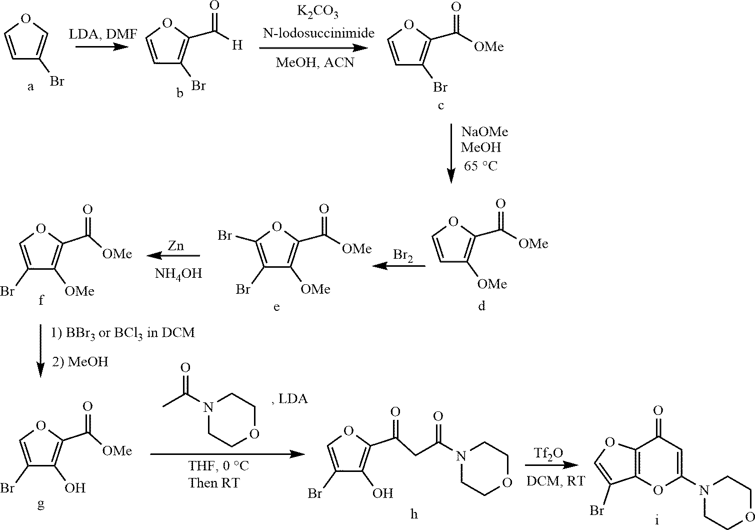

An expanded reaction scheme for introducing substituents at R4 of furan-based compounds of the invention are based on methods described in US20120022059-A1 which are herein incorporated by reference and shown below:

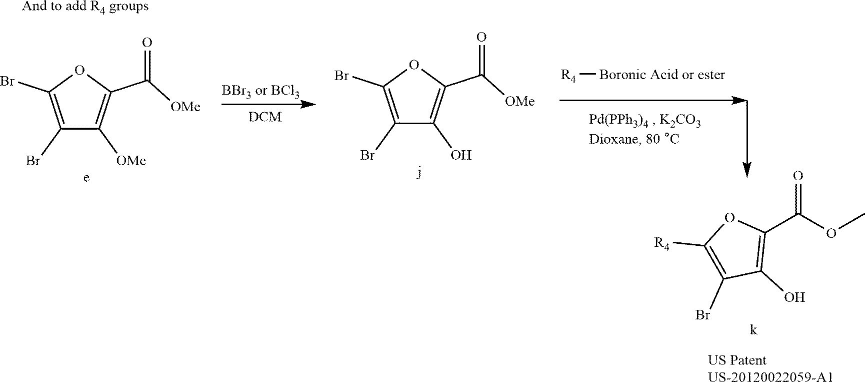

### Scheme for Introducing Substituents at R4 of TP Scaffold Core

The selective introduction of substituents at the R4 position of thiophene containing compounds of the invention is based on the synthesis of molecule “m” (R4 is pyrazole) starting from molecule “1” as disclosed in published US Pat. Application 2016/0287561, the entire contents of which is herein incorporated by reference.

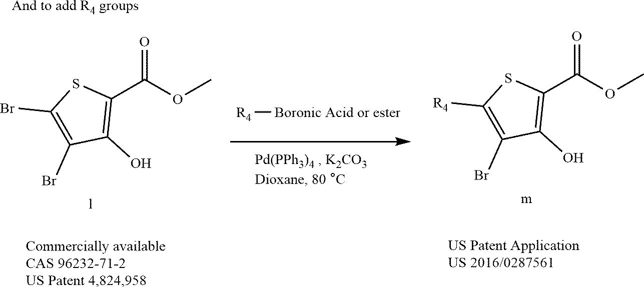

An additional scheme to obtain furanopyranones is shown below using NaN₃ to arrive at the key bromo-hydroxy-furan “g” which can then be used to make intermediate “i” and subsequent elaboration to compounds of the invention:

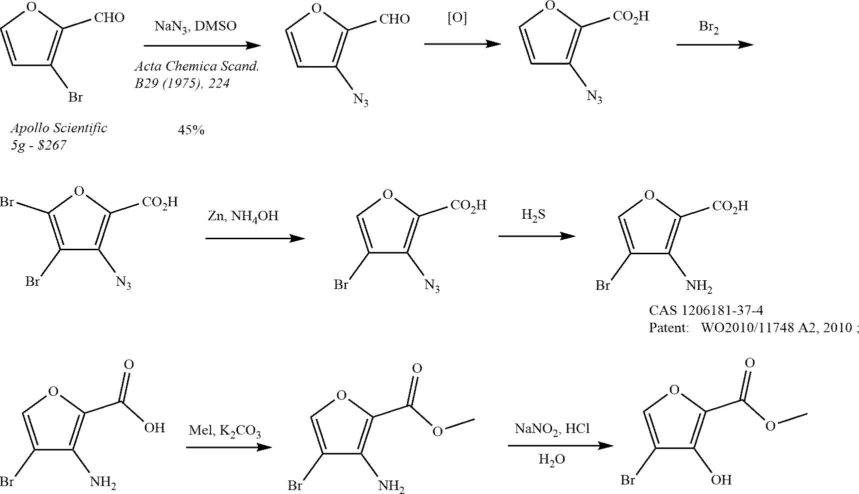

Compounds of the invention having various R2 substituents other than morpholine are made using, for example, acetylated amines, acetylated alcohols, or other methyl ketones in place of the acetyl morpholine. For example, use of acetone in the reaction scheme would give R2 = methyl group. Also, compounds of the invention with various R1 substituents are made using substituted ketones or substituted acetyl morpholine, for example, use of propionylmorpholine would yield R1= methyl group.

The methods of use may utilize compounds or their pharmaceutically acceptable salts, which may contain enhanced levels of naturally occurring stable isotopes in their structure. Some elements like phosphorus and fluorine only exist naturally as a single isotope, with a natural abundance of 100%. However, other elements that may appear in compounds of the invention exist naturally in the abundances listed in the Table 5 below:

TABLE 5

Isotopic abundances.

| Isotope | % nat. abundance | atomic mass |
|---|---|---|
| ¹H | 99.985 | 1.007825 |
| ²H | 0.015 | 2.0140 |
| ¹²C | 98.89 | 12 (definition) |
| ¹³C | 1.11 | 13.00335 |
| ¹⁴N | 99.64 | 14.00307 |
| ¹⁵N | 0.36 | 15.00011 |
| ¹⁶O | 99.76 | 15.99491 |
| ¹⁷O | 0.04 | 16.99913 |
| ¹⁸O | 0.2 | 17.99916 |
| ³²S | 95.0 | 31.97207 |
| ³³S | 0.76 | 32.97146 |
| ³⁴S | 4.22 | 33.96786 |
| ³⁷Cl | 24.23 | |
| ^{g35}Cl | 75.77 | 34.96885 |
| ⁷⁹Br | 50.69 | 78.9183 |
| ^{B1}Br | 49.31 | 80.9163 |

(Source: https ://en.wikipedia.org/wiki/Natural abundance)

The methods of use described herein may utilize compounds intentionally synthesized to contain higher percentages of the minor natural isotope up to 100%. For example, the R2 group of Formula I could be a fully deuterated (²H) morpholino group prepared by substituting in commercially available deuterated morpholine (see http://shop.isotope.com/productdetails.aspx?itemno=DLM-3484-PK for 98% ²H morpholine) for morpholine in the overall synthesis. Other isotopically enriched starting materials and intermediates can also be incorporated by one skilled in the art into the other R groups of Formula 1. Such deuterated pharmaceutical compounds are known to those skilled in the art (e.g., see US 9,676,790 B2 and references therein) and are incorporated by reference herein. Enriched stable isotopic starting materials for preparing isotopically enriched compounds of the invention are available from several vendors including Cambridge Isotope Laboratories Inc. (http://www.isotope.com/index.cfm), Isoflex (https://www.isoflex.com/), and CDN Isotopes (https://cdnisotopes.com/).

### D. Formulations

As an additional aspect of the invention there is provided a pharmaceutical formulation or composition comprising in association with a pharmaceutically acceptable carrier, diluent or excipient, a compound of the invention, e.g., a compound of Formula I (or a pharmaceutically acceptable salt or procompound or conjugate thereof) as provided in any of the descriptions herein for use in a method of the invention. Compositions of the present invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, acacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

Compositions used in the methods of the present invention may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

Compositions used in the methods of the present invention may also be formulated as suppositories, which may contain suppository bases including, but not limited to, cocoa butter or glycerides. Compositions of the present invention may also be formulated for nasal or pulmonary inhalation, which may be in a form including, but not limited to, a solution, suspension, or emulsion that may be administered as a dry powder or in the form of an aerosol using a propellant, such as dichlorodifluoromethane or trichlorofluoromethane. Compositions of the present invention may also be formulated for transdermal administration comprising aqueous or nonaqueous vehicles including, but not limited to, creams, ointments, lotions, pastes, medicated plaster, patch, or membrane.

Compositions used in the methods of the present invention may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions used in the methods of the present invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

Compositions used in the methods of the present invention may also be formulated as a liposome preparation. The liposome preparation can comprise liposomes which penetrate the cells of interest or the stratum corneum, and fuse with the cell membrane, resulting in delivery of the contents of the liposome into the cell. For example, liposomes such as those described in U.S. Pat. No. 5,077,211 of Yarosh et al., U.S. Pat. No. 4,621,023 of Redziniak et al., or U.S. Pat. No. 4,508,703 of Redziniak et al., can be used. Other suitable formulations can employ niosomes. Niosomes are lipid vesicles similar to liposomes, with membranes consisting largely of non-ionic lipids, some forms of which are effective for transporting compounds across the stratum corneum.

The following formulation examples are illustrative only and are not intended to limit the scope of the compounds used in the methods of the invention in any way. The phrase “active ingredient” refers herein to a compound according to Formula I or a pharmaceutically acceptable salt, procompound, conjugate, or solvate thereof.

Formulation 1

Tablet containing the following components:

| Ingredient | Amount (mg/tablet) |
|---|---|
| Active ingredient | 250 |
| Dried starch | 200 |
| Magnesium stearate | 10 |
| Total | 460 mg |

Formulation 2

Capsule containing the following components:

| Ingredient | Amount (mg/tablet) |
|---|---|
| Active ingredient | 60 |
| Dried starch | 44 |
| Magnesium stearate | 1.5 |
| Microcrystalline cellulose | 44 |
| Total | 150 mg |

Parenteral dosage forms for administration to patients by various routes including, but not limited to, subcutaneous, intravenous (including bolus injection), intramuscular, and intra-arterial are also contemplated by the present invention. Parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer’s Injection, Dextrose Injection, Dextrose and Sodium Chloride Injection, and Lactated Ringer’s Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

An example parenteral composition for use in a method of the invention may be intended for dilution with aqueous solution(s) comprising for example 5% Dextrose Injection, USP, or 0.9% Sodium Chloride Injection, USP, prior to administration to a patient, and is an aqueous solution that comprises irinotecan, sorbitol NF powder, and lactic acid, USP, and has a pH of from about 3.0 to about 3.8.

### E. Therapeutic Use

In another aspect of the present invention, a compound or composition of the invention is administered alone or in combination with one or more other agents including but not limited to anti-viral agents such as chloroquine, hydroxychloroquine, Remdesivir, Leronlimab, EIDD-2801, and Ivermectin to a mammal in need thereof including a human to treat or prevent a viral infectious disease including, but not limited to, a COVID-19 coronavirus infection and/or complication thereof by administering a therapeutically effective dose of a compound of Formula I. Other anti-viral agents include drugs classified as entry blockers, nucleoside/nucleoside analogues and nonnucleoside analogues, which interfere with nucleic acid synthesis, IFNs, which inhibit protein synthesis necessary for viral replication, and protease inhibitors (E. De Clercq, J. Clin. Virol., 30, 115-133, 2004). Examples of other antiviral drugs that could be co-administered with a compound of the invention include Amantadine, Rimantadine, Ibalizumab, Enfuvirtide, Vicriviroc, Aciclovir, Valacyclovir, Zidovudine (AZT), Zalcitabine, Cidofovir, Foscarnet, Vidarabine, Ganciclovir, Efavirenz, Nevirapine, Rilpivirine, Etravirine, IFNs, Atazanavir, Fosamprenavir, Lopinavir, Darunavir, Nelfivavir, Indinavir, Saquivavir, and Ritonavir.

Without intending to be bound by theory, it is believed that the therapeutic effectiveness of a compound of the invention involves simultaneous inhibition of at least one BET protein (e.g., BRD2 and BRD4) and at least one kinase (e.g., mTOR and PI3K) with a single molecule. Inhibiting multiple targets with a single drug provides a sophisticated combination therapy for patients resulting in more effective and durable clinical benefits.

In another aspect, the present invention relates to administering a therapeutically effective amount of a compound of the invention to a patient afflicted with a coronavirus infection including but not limited to COVID-19.

In another aspect, the present invention relates to administering a therapeutically effective amount of a compound of the invention to a patient afflicted with COVID-19 and having a pre-existing condition such as but not limited to diabetes, heart disease, chronic kidney disease, obesity, liver disease, hypertension, being 65 years or older, chronic lung disease, asthma, or being immunocompromised.

In another aspect, the present invention relates to administering a therapeutically effective amount of a compound of the invention to a patient who has been or is afflicted with COVID-19 and suffering from one or more complications of the COVID-19 infection including but not limted to acute respiratory failure, pneumonia, acute respiratory distress syndrome (ARDS), lung fibrosis, pulmonary fibrosis, scleroderma, acute liver injury, acute cardiac injury, secondary infection, acute kidney injury, septic shock, disseminated intravascular coagulation, and rhabdomylosis.

In another aspect, the present invention relates to administering a therapeutically effective amount of a compound of the invention to a patient who has been or is infected with a virus causing COVID-19 to treat or prevent one or more complications of the COVID-19 infection including but not limted to acute respiratory failure, pneumonia, acute respiratory distress syndrome (ARDS), lung fibrosis, pulmonary fibrosis, scleroderma, acute liver injury, acute cardiac injury, secondary infection, acute kidney injury, septic shock, disseminated intravascular coagulation, and rhabdomylosis.

In another aspect, the invention relates to a method for inhibiting multiple targets with a single compound of the invention in each cell at the same time wherein the inhibition achieved is superior in a greater percentage of cells than that achieved by a combination of inhibitors of those same targets.

The kinase or bromodomain inhibitory activity of a compound of the invention can be determined by routine methods known to the skilled artisan without undue experimentation, or by procuring relevant analysis by a commercial vendor offering such services. For example, in vitro kinase inhibition (e.g., PI3K inhibition) can be determined by a standard kinase inhibition assay using labeled ATP to determine if a test compound inhibits the transfer of phosphate from ATP to the kinase substrate. In vivo, PI3K inhibition can be determined from target tissue biopsies by standard tissue processing in which cells are disrupted and Western Blot analysis is performed to determine the presence or absence of pAKT (substrate of PI3K) relative to a control sample. PI3K inhibition assays and BTK inhibition assays are known in the art and can be procured commercially through vendors such as Reaction Biology (Malvern, PA). The activity of a compound of the invention as an inhibitor of a bromodomain-containing protein, such as a BET protein, including BRD2, BRD3, BRD4, and/or BRDT, or an isoform or mutant thereof, may be determined in vitro, in vivo, or in a cell line. In vitro assays include assays that determine inhibition of bromodomain-containing proteins. Alternatively, inhibitor binding may be determined by running a competition experiment where a provided compound is incubated with a bromodomain-containing protein, such as a BET protein bound to known ligands, labeled or unlabeled. In vitro bromodomain inhibition assays can be performed using Alpha Screen Technology (Perkin Elmer Life and Analytical Sciences, Shelton, CT). In vivo bromodomain inhibition can be determined indirectly by evaluating the amount of a protein whose gene transcription is influenced or controlled by the bromodomain protein, for example, MYCN protein transcription is controlled by BRD4 (J.E. Delmore et al., Cell 2011, 146, 904-917; A. Puissant, Cancer Discov. 2013, 3, 308-323).

The identification of patients who are in need of treatment for the disorders described herein is within the ability and knowledge of one skilled in the art. Certain of the methods for identification of patients who are at risk of developing the above disorders which can be treated by a method of the invention are appreciated in the medical arts including, for example, family history and the presence of risk factors associated with development of the disease state.

Assessing the efficacy of a treatment in a patient may include determining the pretreatment extent of a disorder by methods known in the art then administering a therapeutically effective amount of a compound of the invention, to the patient. Following an appropriate period of time after administration (e.g., 1 day, 1 week, 2 weeks, one month, six months), the extent of the disorder is again determined. The extent or invasiveness of the disorder may be determined periodically throughout treatment. For example, the extent or invasiveness of the disorder may be assessed every few hours, days or weeks to assess the further efficacy of the treatment. A decrease in extent or invasiveness of the disorder would indicate that the treatment is efficacious. The methods described may be used to screen or select patients that may benefit from treatment with a compound of the invention.

### F. Administration and Dosage

Compounds or compositions of Formula I for use in a therapeutic method of the present invention can be administered in any manner including but not limited to orally, parenterally, sublingually, transdermally, rectally, transmucosally, topically, pulmonarily, nasally, or bucally. Parenteral administration includes but is not limited to intravenous, intraarterial, intraperitoneal, subcutaneous, intramuscular, intrathecal, and intraarticular. Compounds or compositions of the invention may also be administered via slow controlled i.v. infusion or by release from an implant device.

A therapeutically effective amount of a compound of Formula I for use in a method of the invention varies with the nature and severity of the condition or infection being treated, the length of treatment time desired, the age and the condition of the patient, and is ultimately determined by the attending physician. In general, however, doses employed for adult human treatment typically are in a range of about 0.001 mg/kg to about 200 mg/kg per day, or about 1 µg/kg to about 100 µg/kg per day. The desired dose may be conveniently administered in a single dose, or as multiple doses administered at appropriate intervals, for example, as two, three, four or more sub-doses per day. Multiple doses over a 24-hour period may be desired or required.

A number of factors may lead to the compounds of Formula I being administered according to the methods of the invention over a wide range of dosages. When given in combination with other therapeutic agents, compounds of the present invention may be provided at relatively lower dosages. The dosage of a compound of Formula I according to the methods of the present invention may be at any dosage including, but not limited to, about 1 µg/kg, 25 µg/kg, 50 µg/kg, 75 µg/kg, 100 µg/kg, 125 µg/kg, 150 µg/kg, 175 µg/kg, 200 µg/kg, 225 µg/kg, 250 µg/kg, 275 µg/kg, 300 µg/kg, 325 µg/kg, 350 µg/kg, 375 µg/kg, 400 µg/kg, 425 µg/kg, 450 µg/kg, 475 µg/kg, 500 µg/kg, 525 µg/kg, 550 µg/kg, 575 µg/kg, 600 µg/kg, 625 µg/kg, 650 µg/kg, 675 µg/kg, 700 µg/kg, 725 µg/kg, 750 µg/kg, 775 µg/kg, 800 µg/kg, 825 µg/kg, 850 µg/kg, 875 µg/kg, 900 µg/kg, 925 µg/kg, 950 µg/kg, 975 µg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

The present invention has multiple aspects, illustrated by the following non-limiting examples. The examples are merely illustrative and do not limit the scope of the invention in any way.

### EXAMPLES

### Example 1. Compound 0 Blocks COVID-19 Infectivity of Human Hela-ACE2 Transduced Cells in Vitro

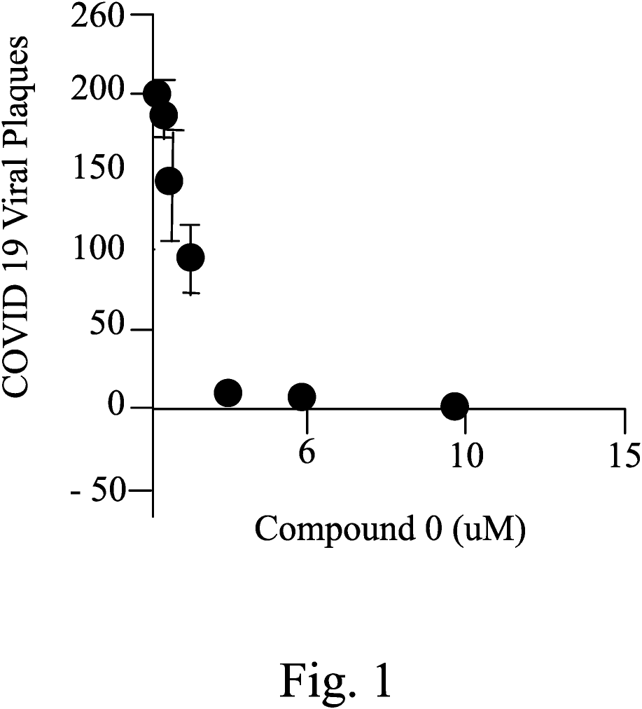

The anti-viral activity of Compound 0 was compared to Remdesivir in vitro. A graph of COVID-19 viral plaques versus Compound 0 concentration allows a calculation of the IC50 value. The response to Compound 0 was found to be dose dependent in this assay system (See FIG. 1). The IC50 for Compound 0 inhibition of SARS-CoV-2 in this assay system was found to be 900 nM which is well within the therapeutic and safety range noted for Compound 0 in multiple animal studies. These results indicate Compound 0 blocks COVID-19 infectivity at doses achievable in mammalian systems. Notably, at 2.4 µM concentration of Compound 0 the inhibition is 94%. At these effective anti-viral concentrations, no cellular cytotoxicity is noted in this assay. For comparison in this assay of viral plaques versus inhibitor concentration the Remdesivir IC50 is 100 nM.

### Example 2. Compound 0 Inhibits Collagen Production in Fibroblasts From Human Scleroderma Patients and Fibrosis in Rodent Model for Lung Fibrosis

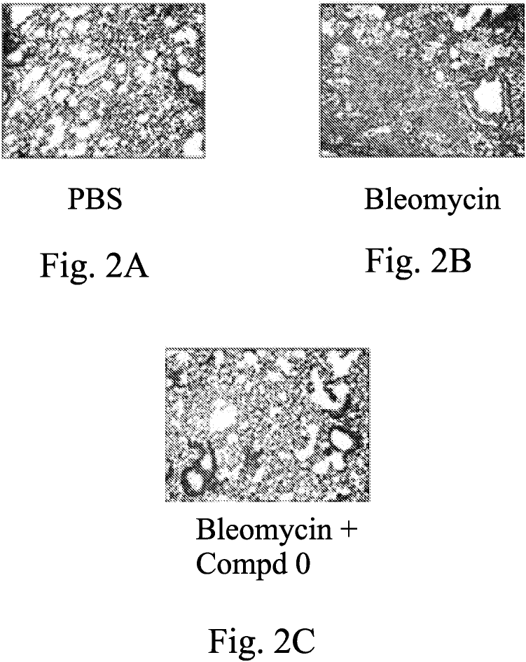

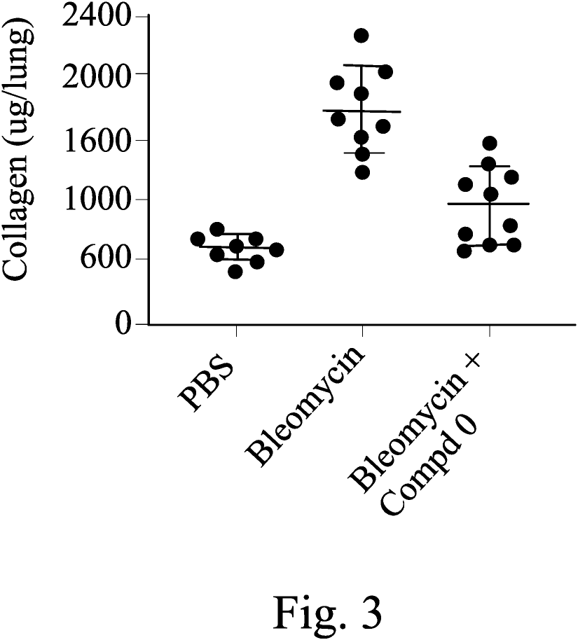

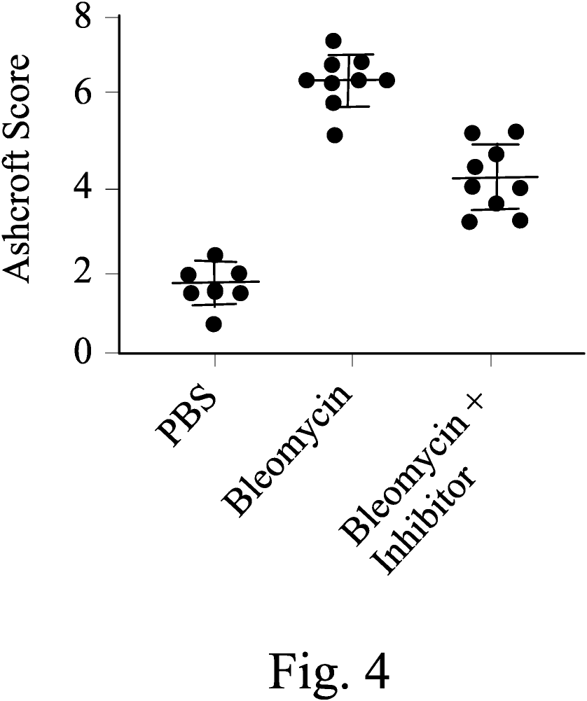

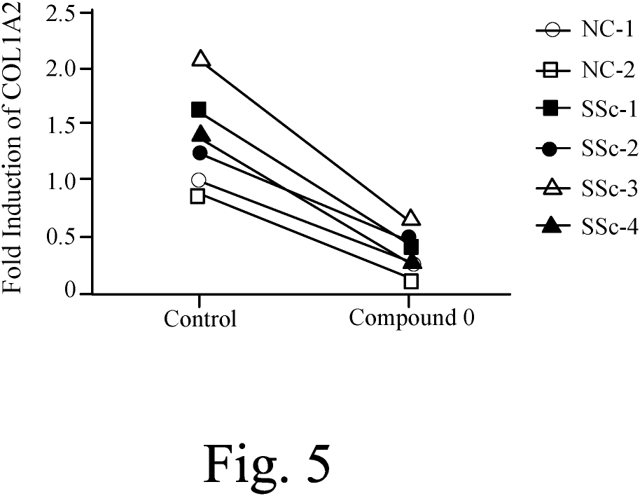

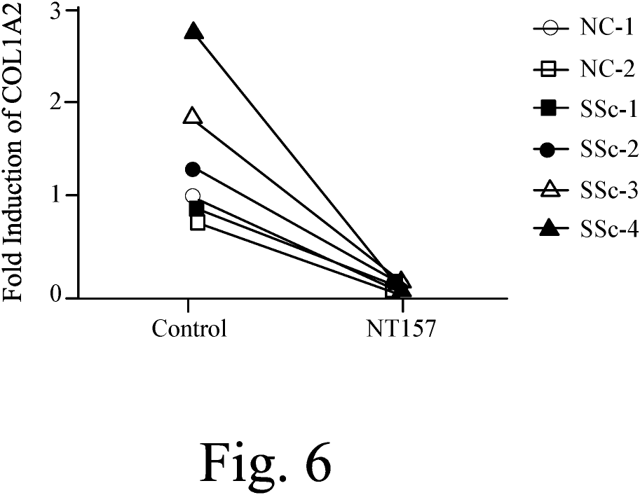

Bleomycin-induced lung fibrosis is the most frequently used rodent model for lung fibrosis, and inflammatory and fibrotic events similar to those seen in human pulmonary fibrosis (Am J Respir Cell Mol Biol. 2013 Aug; 49(2): 167-179; https://www.atsjournals.org/doi/full/10.1165/rcmb.2013-0094TR; PMID: 23526222). Systemic sclerosis (SSc) is manifested by vasculopathy, immune dysregulation and fibrosis of the skin and certain internal organs, especially lungs (Nat Commun. 2014; 5: 5797.; https://www.nature.com/articles/ncomms6797; PMID: 25504335). Friend leukemia integration 1 (Fli1) is a potent repressor of the type I collagen gene. Mice carrying the Fli1 deletion manifest greatly increased lung fibrosis when administered intra-tracheal bleomycin. Bleomycin was administered intra-tracheally to mice (0.4 mg/kg) and no drug was given during the inflammatory phase (8 days). Then, compound 0 (50 mg/kg) was administered three times during the ensuing fibrotic phase (day 9, 12, 15). The animals were sacrificed (day 21) and compound 0 was found to be remarkably effective in preventing the development of fibrosis both by Masson Trichrome staining of tissue sections (FIGS. 2A-2C), tissue collagen content (FIG. 3) and by the Ashcroft clinical score of the mice (FIG. 4). Overall, these results demonstrate that compound 0 blocks pulmonary fibrosis in an acute lung injury model in vivo. Further evidence of the impact of compound 0 on fibrosis prevention was determined by conducting an in vitro study of collagen 1A2 production in cultured fibroblasts from patients with scleroderma and controls in the presence and absence of compound 0 (20 mM/mL) or insulin receptor substrate 1 (IRS-1) inhibitor (NT157). IRS-1 is a component of the insulin signaling pathway which is involved in the increased collagen production by cells carrying the Fli1 deletion. Compound 0 inhibited collagen 1A2 production (see FIGS. 5 and 6)

### Example 3. Anti-Viral Activity of Compound 0 and Remdesivir Alone and in Combination

### Cell Lines

Vero-STAT1: Vero-STAT1 knockout cells were obtained from ATCC (CCL-81-VHG™) and cultured in DMEM containing 10% fetal bovine serum, 2 mM L-glutamine, penicillin (100 units/ml), streptomycin (100 units/ml), and 10 mM HEPES. STAT1 is a transcription factor essential for interferon mediated host cell anti-viral response. Hence, the STAT1 knockout cells are highly susceptible to viral infection due to the absence of cellular anti-viral response, and serve as a positive control in this study.

UNCN1T: UNCN1T cells were obtained from Kerafast (cat# ENC011) and were cultured using BEGM media (Bronchial Epithelial Cell Growth Medium; Lonza: cat# CC-3170) in FNC coated plates (Athena Enzyme Systems; cat# 0407). UNCN1T is a human bronchial epithelial cell line expected to better recapitulate the in vivo pathogenesis of SARS-CoV-2 in lung, and serves as a good model for in-vitro efficacy study of anti-viral compounds against SARS-CoV-2.

### Cell Procedures and Calculations

Cells were incubated at 37° C. with 5% CO₂. 24 hours before infection 20,000 cells/well were seeded in 96 well plates. Different concentrations of Compound 0 and Remdesivir (100 µM, 10 µM, 5 µM, 1 µM, 0.1 µM, 0.01 µM and 0.001 µM) were added to the cells 2 hours before infection. The cells were infected with 0.1 MOI of SARS-CoV-2 (Isolate USA-WI1/2020; BEI cat# NR-52384) using Opti-MEM® I reduced serum medium (Thermo Fisher, Cat#31985062) and incubated for 1 hour at 37° C. with 5% CO₂. For positive control, cells were treated with the same volume of DMSO equivalent to the volume of drugs added. Mock infected cells received only Opti-MEM° I reduced serum medium. At the end of incubation, virus inoculum was removed, cells were washed with 1X PBS 3 times and fresh media was added supplemented with the same concentration of drugs. Culture supernatant was collected at 24 hrs and 48 hrs post-infection and SARS-CoV-2 viral load was quantified using RT-QPCR with primer probes targeting the E gene of SARS-CoV-2 using PrimeDirect Probe RT-qPCR Mix (TaKaRa Bio USA, Inc) and Applied Biosystems QuantStudio3 real-time PCR system (Applied Biosystems, Waltham, MA, USA) per manufacturer’s instructions. Primers and probes used for SARS-CoV-2 RNA quantification were as follows: E_Sarbeco_F1: 5′ - ACAGGTACGTTAATAGTTAATAGCGT - 3′ (400 nM), E_Sarbeco_R2: 5′ - ATATTGCAGCAGTACGCACACA - 3′ (400 nM) and E_Sarbeco _P1: 5′ -FAM-ACACTAGCCATCCTTACTGCGCTTCG - BHQ1 - 3′ (200 nM) as recommended by WHO. The SARS-CoV-2 genome equivalent copies were calculated using quantitative PCR (qPCR) control RNA from heat-inactivated SARS-CoV-2, isolate USA-WA1/2020 (BEI; cat# NR-52347). The percentage inhibition of SARS-CoV-2 replication in Compound 0 and Remdesivir treated wells were calculated with respect to viral concentration in positive control wells that were treated with DMSO (considered 0% inhibition) and negative control wells (uninfected cells). IC50 values were calculated using four parameter variable slope sigmoidal dose-response models using Graph Pad Prism 8.0 software. Cytopathic effect (CPE) was determined using the CellTiter-Glo luminescent cell viability assay (Promega; Madison, WI; cat# G9243) as per manufacturer’s instructions (In this assay, the number of viable cells in culture was determined by quantifying ATP, which indicates the presence of metabolically active cells. Luminesence readout is directly proportional to the number of viable cells in culture a reflection of antiviral cellular protection conferred by each targeted agent in vitro. RLU values were plotted against log drug concentrations after normalization with RLU values from blank wells having no cells. Antiviral activity was determined by the degree of inhibition of viral replication.

### Combination Procedures

To determine possible combinational effects of Compound 0 and remdesivir against SARS-CoV-2, we tested 5 (remdesivir) X 4 (Compound 0) dose combinations using SARS-CoV-2 infected VERO-STAT1 KO and UNCN1T cells. The percentage inhibition of viral replication for each dose combination was determined by QPCR as described above. In brief, the VERO-STAT1 KO and UNCN1T cells were treated with different combination doses of Compound 0 and Remdesivir, infected with 0.1 multiplicity of infection (MOI) of SARS-CoV-2. 24 hours post-infection culture supernatant was collected and SARS-CoV-2 viral load was quantified using RT-QPCR as described above. Then the percent inhibition of viral replication for 1:1 fixed dose combination of the drugs was used to generate CI-Fa, isobologram and dose-response plots. The combination index (CI) was calculated using the multiple drug effect equation developed by Chou and Talalay using the CompuSyn algorithm (https://www.combosyn.com). A CI values of <1 indicates synergy, values >1 indicate antagonism, and values equal to 1 indicate additive effects [see for example: T. C. Chou, Drug combination studies and their synergy quantification using the Chou-Talalay method. Cancer Res 70, 440-446 (2010); T. C. Chou, Theoretical basis, experimental design, and computerized simulation of synergism and antagonism in drug combination studies. Pharmacol Rev 58, 621-681 (2006); T. C. Chou, P. Talalay, Quantitative analysis of dose-effect relationships: the combined effects of multiple drugs or enzyme inhibitors. Adv Enzyme Regul 22, 27-55 (1984)].

### Vero-STAT1 Results

In Vero-STAT1 knockout cells, Compound 0 and remdesivir both show substantial reduction in SARS-CoV-2 viral load 24 hours and 48 hours post-infection. Based on SARS-CoV-2 viral loads in culture supernatant, Compound 0 showed potential anti-viral activity with an IC50 value of 1.02 µM and 3.22 µM 24 hrs and 48 hrs post-infection, while the reference drug remdesivir showed an IC50 values of 1.03 µM and 0.76 µM at 24 hrs and 48 hrs post infection. Moreover, based on the dose response curve by cytopathic effect (CPE), Compound 0 shows anti-viral activity with an IC50 value of 1.8 µM and 4.4 µM 24 hrs and 48 hrs post-infection, while remdesivir showed an IC50 values of 2.24 µM and 4.60 µM at 24 hrs and 48 hrs post infection.

### UNCN1T Results

In UNCN1T cells Compound 0 and remdesivir both showed substantial reduction in SARS-CoV-2 viral load 24 hours and 48 hours post-infection. Based on SARS-CoV-2 viral loads in the culture supernatant, Compound 0 showed potential anti-viral activity with an IC50 value of 1.52 µM and 1.58 µM 24 hrs and 48 hrs post infection, while remdesivir showed an IC50 values of 1.06 µM and 2.75 µM at 24 hrs and 48 hrs post-infection. On the other hand, based on the dose response curve by cytopathic effect (CPE), Compound 0 showed anti-viral activity with an IC50 value of 0.25 µM 24 hrs post-infection, while remdesivir showed an IC50 value of 0.22 µM at 24 hrs post-infection.

### Combination Results

Table 6 below indicates the median effective dose of Compound 0, remdesivir and their fixed dose combinations, and demonstrates a clear beneficial effect of combining Compound 0 with remdesivir.

TABLE 6

Median effective dose of individual compounds and their combination
VERO STAT1 KO Cells

| Drug/Combo | Median Effective Dose (µM) |
|---|---|
| Compound 0 | 2.617 |
| Remdesivir | 0.533 |
| Combination | 0.452 |
| UNCN1T Cells | |
| Drug/Combo | Median Effective Dose (µM) |
| Compound 0 | 1.836 |
| Remdesivir | 0.272 |
| Combination | 0.145 |

Table 7 below shows the CI values at ED50, ED75, ED90 and ED95 in VERO-STAT1 knock out and UNCN1T cells, which suggests a moderate to strong synergistic effect of the drug combinations. In VERO-STAT1 KO cells at Fa 0.5, a dose reduction index (DRI) of 11.56 was obtained for Compound 0 and 2.35 for remdesivir respectively. On the other hand, in UNCN1T cells, a dose reduction index (DRI) of 25.33 was obtained for Compound 0 and 3.75 for remdesivir, respectively.

TABLE 7

CI values at different simulated effective dose of fixed dose combination of Compound 0 and remdesivir
CI values

| Cell Type | ED50 | ED75 | ED90 | ED95 |
|---|---|---|---|---|
| VERO-STAT1-KO cells | 0.5105 | 3.75E-05 | 1.44E-08 | 7.10E-11 |
| UNCN1T cells | 0.3054 | 0.0173 | 9.91E-04 | 1.42E-04 |

### Example 4. in Vitro Potency (IC50_nM) of Compounds of the Invention Against BRD, PI3K, CDK, BTK, Syk, and mTOR Targets

IC50 inhibition data against the indicated targets were determined for compounds of the invention using standard in vitro assays (Tables 8 and 9).

TABLE 8

IC50 inhibition (nM) by compounds of the invention.

| Compd | BRD4-1 | BRD4-2 | PI3K alpha | PI3K beta | PI3K gamma | CDK4 cyclinD1 | CDK6 cyclinD1 | BTK | BTK C481S | mTOR |
|---|---|---|---|---|---|---|---|---|---|---|
| 0 | + | ++ | + | + | + | | | ++++ | ++++ | + |
| 6 | + | + | + | + | +++ | + | + | | | +++ |
| 10 | ++ | +++ | +++ | ++ | +++ | + | + | | | |
| 9 | + | ++ | ++++ | ++++ | ++++ | + | + | | | |
| 8 | + | + | + | + | ++ | + | + | | | |
| 12 | ++++ | ++++ | +++ | + | +++++ | +++ | +++++ | | | |
| 11 | + | +++ | + | + | +++ | +++++ | +++++ | | | |
| 2 | + | + | + | + | + | | | | | |
| 13 | ++ | ++ | + | + | ++ | +++++ | +++++ | | | |
| 14 | +++++ | ++++ | + | + | + | ++++ | ++++ | | | |
| 5 | + | +++++ | + | +++ | + | | | | | |
| 30 | +++ | + | + | + | ++ | | | ++++ | +++++ | |
| 31 | ++ | +++ | + | + | +++ | | | ++ | +++++ | |
| 15 | ++++ | ++++ | + | + | +++ | +++ | ++ | | | |
| 17 | +++ | ++++ | + | + | ++ | + | + | | | |
| 16 | + | + | + | + | +++ | + | + | | | |
| 18 | +++ | ++ | ++ | + | +++ | + | + | | | |
| 4 | + | +++ | ++++ | +++ | ++++ | | | | | |
| 19 | +++ | +++ | ++ | + | +++ | + | + | | | |
| 20 | ++++ | +++++ | +++ | +++ | +++++ | ++++ | +++++ | | | |
| 21 | +++ | +++ | +++ | +++ | ++++ | + | ++ | | | |
| 3 | + | +++ | +++ | +++ | ++++ | | | | | |
| 1 | + | ++++ | +++++ | ++++ | +++++ | | | | | |
| 27 | ++ | +++ | +++++ | +++++ | +++++ | | | +++ | ++++ | |
| 32 | + | + | + | + | + | | | + | ++++ | |
| 33 | + | +++ | + | + | + | | | ++ | +++++ | |
| 22 | +++++ | +++ | ++ | + | ++++ | + | + | | | |
| 34 | +++ | ++++ | +++ | +++ | ++++ | | | +++++ | +++++ | |
| 35 | +++ | ++ | + | + | +++ | | | ++++ | ++++ | |
| 7 | ++++ | +++ | +++ | ++++ | ++++ | + | + | | | +++ |
| 23 | ++ | +++ | + | + | + | + | + | | | |
| 26 | + | +++ | ++++ | +++ | +++++ | | | + | ++++ | |
| 28 | +++ | ++++ | ++++ | +++ | +++++ | | | + | +++ | |
| 36 | + | ++ | + | + | +++ | | | +++ | +++ | |
| 29 | ++ | +++ | ++++ | +++ | ++++ | | | + | ++++ | |
| 37 | ++ | ++ | + | + | ++ | | | ++++ | +++++ | |
| 38 | + | ++ | + | + | +++ | | | +++++ | +++++ | |
| 39 | + | + | + | + | +++ | | | ++++ | +++++ | |
| 40 | + | + | + | + | +++ | | | +++++ | ++++ | |
| 41 | + | + | + | + | +++ | | | +++ | ++++ | |
| 24 | | | + | + | +++ | +++++ | | | | |
| 25 | + | + | + | + | +++ | + | + | | | |
| 44 | + | + | + | + | + | | | + | ++++ | |
| 45 | + | + | + | + | + | | | + | +++++ | |
| 46 | + | + | + | + | + | | | + | +++++ | |
| 47 | + | ++ | + | + | + | | | + | +++++ | |
| 48 | + | + | + | + | + | | | + | +++++ | |
| 49 | + | +++ | + | + | + | | | + | ++++ | |
| 51 | +++ | + | + | + | ++ | | | +++ | ++++ | |
| 50 | + | + | + | + | + | | | + | ++++ | |

+++++ means IC50s > 45,000 nM
++++ means IC50s between 10,000 nM and 45,000 nM
+++ means IC50s between 1,000 nM and 10,000 nM
++ means IC50s between 500 nM and 1,000 nM
+ means IC50s < 500 nM
No entry means not determined

TABLE 9

IC50 inhibition (nM) by compounds of the invention.

| Compd | BRD4-1 | BRD4-2 | PI3K alpha | PI3K delta | PI3K gamma | CDK4 cyclinD1 | CDK6 cyclinD1 | Syk |
|---|---|---|---|---|---|---|---|---|
| 52 | +++++ | +++++ | ++ | +++ | ++++ | ++++ | + | + |
| 53 | ++++ | +++++ | +++ | +++ | +++++ | +++ | + | + |
| 56 | +++ | +++++ | +++ | +++ | ++++ | +++ | ++ | + |
| 54 | +++ | +++ | + | + | +++ | +++++ | +++++ | + |
| 55 | +++ | +++++ | +++++ | +++ | +++++ | +++ | + | + |
| 57 | +++ | +++ | ++ | ++ | ++++ | + | + | + |
| 58 | +++ | +++ | ++ | + | ++++ | +++++ | +++++ | +++++ |
| 59 | +++ | +++ | +++ | +++ | ++++ | +++ | +++ | ++ |
| 5-1 | ++++ | ++++ | + | + | + | | | |
| 5-2 | ++ | +++ | + | + | ++ | | | |
| 5-3 | + | ++ | ++ | + | +++ | | | |
| 5-4 | +++ | ++++ | + | + | + | | | |
| 25-1 | +++++ | +++++ | ++++ | +++ | +++++ | + | + | |
| 25-2 | + | + | + | + | +++ | + | + | |
| 25-3 | + | ++ | + | + | +++ | + | + | |

+++++ means IC50s > 45,000 nM
++++ means IC50s between 10,000 nM and 45,000 nM
+++ means IC50s between 1,000 nM and 10,000 nM
++ means IC50s between 500 nM and 1,000 nM
+ means IC50s < 500 nM
No entry means not determined

1. A method for the treatment or prevention of a coronavirus infection in a mammal in need thereof comprising administering a therapeutically effective amount of a compound of Formula I or a pharmaceutically acceptable salt thereof, 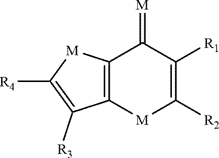
wherein M is independently oxygen (O) or sulfur (S);
R1 is selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from R1, morpholine, thiomorpholine, or piperazine;
R3 is selected from R1; and
R4 is selected from R1.

2. A method for the treatment or prevention of a coronavirus infection in a mammal as in claim 1, wherein a compound of Formula I is further characterized by Formula II or a pharmaceutically acceptable salt thereof, 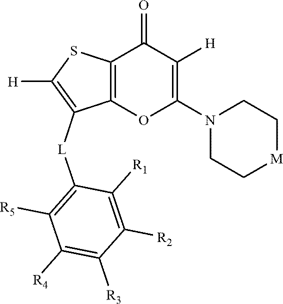
wherein M is independently oxygen (O) or sulfur (S) or N-R₁;
L is null or acetylenic;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from R1,
R3 is selected from R1;
R4 is selected from R1;
R5 is selected from R1; and
where R1-R5 may independently contain varying amounts of isotopic substitution.

3. A method for the treatment or prevention of a coronavirus infection in a mammal as in claim 1, wherein a compound of Formula I is further characterized by a compound of Formula III or a pharmaceutically acceptable salt thereof, 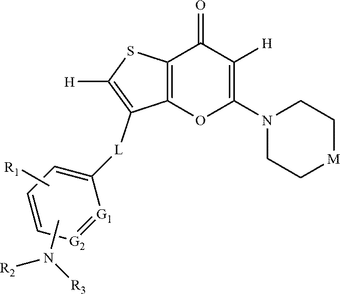
wherein M is independently oxygen (O) or sulfur (S) or N-R₁;
L is null or acetylenic;
G1 and G2 are independently selected from CH and N;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from hydrogen, methyl, or R3;
R3 is 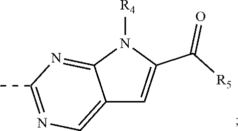
R4 is selected from a straight or branched or cyclic C1 to C6 aliphatic chain;
R5 is selected from hydroxyl, NH₂, NH(CH₃), N(CH₃)₂, NH-OH, NH-OCH₃; and
where R1-R5 may independently contain varying amounts of isotopic substitution.

4. A method for the treatment or prevention of a coronavirus infection in a mammal as in claim 1, wherein a compound of Formula I is further characterized by a compound of Formula IV or a pharmaceutically acceptable salt thereof, 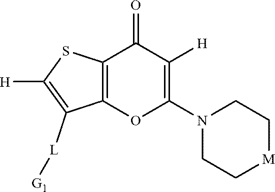
wherein M is independently oxygen (O) or sulfur (S) or N-R₁;
L is null or acetylenic;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
G1 is selected from 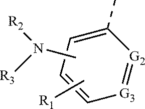
or 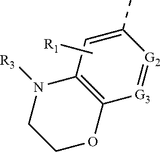
or 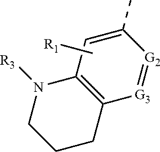
G2 and G3 are independently selected from CR1 and N;
R2 is selected from hydrogen, methyl, ethyl, 2-hydroxyethyl, 2-methoxyethyl, or R3;
R3 is selected from —S(O)(O)—R4 or —C(O)—R4;
R4 is selected from 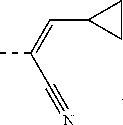
C2 to C10 alkyl, carbocyclic, alkenyl, alkynyl chain or
cycloalkenyl; and
where R1-R4 may independently contain varying amounts of isotopic substitution.

5. A method for the treatment or prevention of a coronavirus infection in a mammal as in claim 1, wherein a compound of Formula I is further characterized by a compound of Formula V or a pharmaceutically acceptable salt thereof, 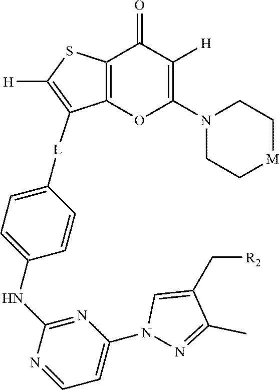
wherein M is independently oxygen (O) or sulfur (S) or N-R₁;
L is null or acetylenic;
R1 is independently selected from H, methyl, —C(O)OC(CH₃)₃;
R2 is selected from 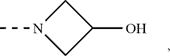 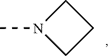 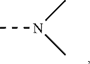 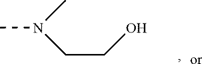 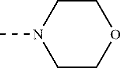
and;
where R1-R2 may independently contain varying amounts of isotopic substitution.

6. A method as in claim 1 wherein said coronavirus is a SARS-CoV-2 infection and said mammal is a human patient.

7. A method as in claim 6 wherein said compound is selected from Compounds 0, 1, 2, 3, 4, 5, 5-1, 5-2, 5-3, 5-4, 6, 7, 8, 9, 10, 11, 12, 13, 14, 15, 16, 17, 18, 19, 20, 21, 22, 23, 24, 25, 25-1, 25-2, 25-3, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, and 59.

8. A method as in claim 7 wherein said patient has a pre-existing condition selected from diabetes, heart disease, chronic kidney disease, obesity, liver disease, hypertension, being 65 years or older, chronic lung disease, asthma, or being immunocompromised.

9. A method as in claim 7 wherein said patient develops one or more complications associated with COVID-19 illness selected from acute respiratory failure, pneumonia, acute respiratory distress syndrome (ARDS), lung fibrosis, scleroderma, acute liver injury, acute cardiac injury, secondary infection, acute kidney injury, septic shock, disseminated intravascular coagulation, and rhabdomylosis.

10. A method as in claim 7 wherein said compound is co-administered with an agent selected from anti-viral agents, chloroquine, hydroxychloroquine, Remdesivir, Leronlimab, EIDD-2801, and Ivermectin.

11. A method as in claim 7 wherein said treatment or prevention occurs by inhibiting at least one member of the BET family and at least one kinase.

12. A method as in claim 11 wherein said member of the BET family is selected from BRD2 and BRD4 and said at least one kinase is selected from mTOR and PI3K.

13. A method as in claim 10 wherein said anti-viral agent is selected from entry blockers, nucleoside/nucleoside analogues and nonnucleoside analogues, IFNs, and protease inhibitors.

14. A method as in claim 13 wherein said anti-viral agent is selected from Amantadine, Rimantadine, Ibalizumab, Enfuvirtide, Vicriviroc, Aciclovir, Valacyclovir, Zidovudine (AZT), Zalcitabine, Cidofovir, Foscarnet, Vidarabine, Ganciclovir, Efavirenz, Nevirapine, Rilpivirine, Etravirine, IFNs, Atazanavir, Fosamprenavir, Lopinavir, Darunavir, Nelfivavir, Indinavir, Saquivavir, and Ritonavir.

15. A method for the treatment or prevention of a complication arising from a coronavirus infection in a mammal in need thereof comprising administering a therapeutically effective amount of a compound of Formula I or a pharmaceutically acceptable salt thereof, 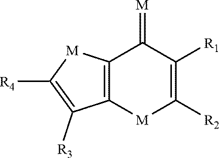
wherein M is independently oxygen (O) or sulfur (S);
R1 is selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from R1, morpholine, thiomorpholine, or piperazine;
R3 is selected from R1; and
R4 is selected from R1.

16. A method as in claim 15 wherein said complication is selected from acute respiratory failure, pneumonia, acute respiratory distress syndrome (ARDS), lung fibrosis, scleroderma, acute liver injury, acute cardiac injury, secondary infection, acute kidney injury, septic shock, disseminated intravascular coagulation, and rhabdomylosis.

17. A method as in claim 16 wherein Formula I is further characterized by Formula II 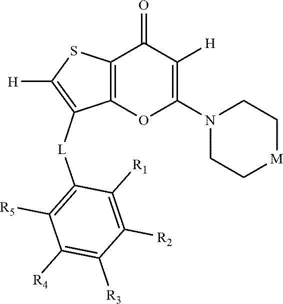
wherein M is independently oxygen (O) or sulfur (S) or N-R₁;
L is null or acetylenic;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from R1,
R3 is selected from R1;
R4 is selected from R1;
R5 is selected from R1; and
where R1-R5 may independently contain varying amounts of isotopic substitution.

18. A method as in claim 16 wherein Formula I is further characterized by Formula III 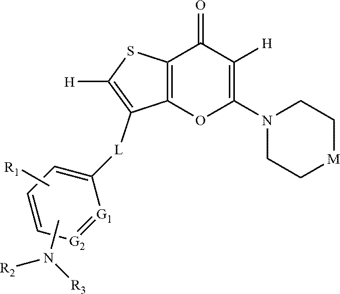
wherein M is independently oxygen (O) or sulfur (S) or N-R₁;
L is null or acetylenic;
G1 and G2 are independently selected from CH and N;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
R2 is selected from hydrogen, methyl, or R3;
R3 is 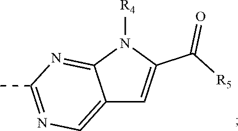
R4 is selected from a straight or branched or cyclic C1 to C6 aliphatic chain;
R5 is selected from hydroxyl, NH₂, NH(CH₃), N(CH₃)₂, NH-OH, NH-OCH₃; and
where R1-R5 may independently contain varying amounts of isotopic substitution.

19. A method as in claim 16 wherein Formula I is further characterized by Formula IV 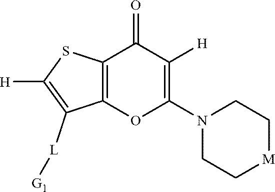
wherein M is independently oxygen (O) or sulfur (S) or N-R₁;
L is null or acetylenic;
R1 is independently selected from H, halogen, alkyl, alkenyl, alkynyl, carbocycle, aryl, heterocycle, heteroaryl, formyl, nitro, cyano, amino, carboxylic acid, carboxylic ester, carboxyl amide, reverse carboxyamide, substituted alkyl, substituted alkenyl, substituted alkynyl, substituted carbocycle, substituted aryl, substituted heterocycle, substituted heteroaryl, phosphonic acid, phosphinic acid, phosphoramidate, phosphonic ester, phosphinic ester, ketone, substituted ketone, hydroxamic acid, N-substituted hydroxamic acid, O-substituted hydroxamate, N- and O- substituted hydroxamate, sulfoxide, substituted sulfoxide, sulfone, substituted sulfone, sulfonic acid, sulfonic ester, sulfonamide, N-substituted sulfonamide, N,N-disubstituted sulfonamide, boronic acid, boronic ester, azo, substituted azo, azido, nitroso, imino, substituted imino, oxime, substituted oxime, alkoxy, substituted alkoxy, aryloxy, substituted aryloxy, thioether, substituted thioether, carbamate, substituted carbamate;
G1 is selected from 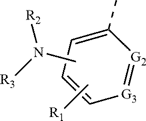
or 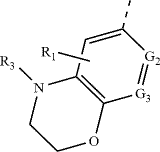
or 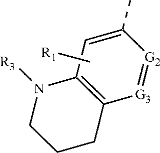
G2 and G3 are independently selected from CR1 and N;
R2 is selected from hydrogen, methyl, ethyl, 2-hydroxyethyl, 2-methoxyethyl, or R3;
R3 is selected from —S(O)(O)—R4 or —C(O)—R4;
R4 is selected from 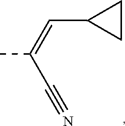
C2 to C10 alkyl, carbocyclic, alkenyl, alkynyl chain or cycloalkenyl; and
where R1-R4 may independently contain varying amounts of isotopic substitution.

20. A method as in claim 16 wherein Formula I is further characterized by Formula V 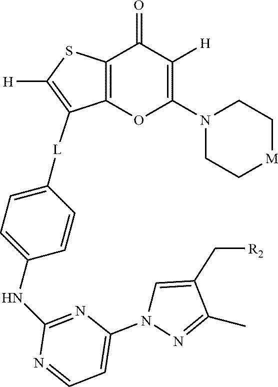
wherein M is independently oxygen (O) or sulfur (S) or N-R₁;
L is null or acetylenic;
R1 is independently selected from H, methyl, —C(O)OC(CH₃)₃;
R2 is selected from 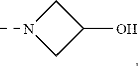 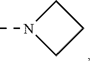 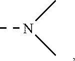 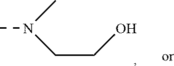 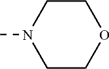
and;
where R1-R2 may independently contain varying amounts of isotopic substitution.
